(19) United States
(12) Patent Application Publication
(10) Pub. No.: US 2017/0204352 A1
(43) Pub. Date: Jul. 20, 2017

(54) Detergent Composition

(71) Applicant: Novozymes A/S, Bagsvaerd (DK)

(72) Inventors: Aihua Yan, Beijing (CN); Nickolass Bitsch Shack, Copenhagen (DK)

(73) Assignee: Novozymes A/S, Bagsvaerd (DK)

(21) Appl. No.: 15/129,620

(22) PCT Filed: Mar. 25, 2015
(86) PCT No.: PCT/CN2015/075033

§ 371 Date: Sep. 27, 2016

(30) Foreign Application Priority Data
Apr. 1, 2014 (CN) PCT/CN2014/074532
Jul. 21, 2014 (CN) PCT/CN2014/082616

(51) Int. Cl. C11D 3/386; C12N 9/54; C12N 9/24; C11D 11/00; C12N 9/28

(52) U.S. Cl. CPC C11D 3/386; C11D 3/38636; C11D 11/0017; C11D 11/0023; C12N 9/2417; C12N 9/2491; C12N 9/54; C12Y 302/01001; C12Y 302/01025

(57) ABSTRACT

A detergent composition comprises a detergent adjunct ingredient and two or more enzymes. The use of two or more enzymes for releasing or removing soil or stains from an item and a cleaning or laundering method are disclosed.

### REFERENCE TO A SEQUENCE LISTING

This application contains a Sequence Listing in computer readable form, which is incorporated herein by reference.

### FIELD OF THE INVENTION

The present invention concerns a detergent composition comprising a detergent adjunct ingredient and two or more enzymes, the use of two or more enzymes for releasing or removing soil or stains from an item and a cleaning or laundering method.

### BACKGROUND OF THE INVENTION

Household cleaning is a daily worldwide activity, which means that billions of people use detergent to clean their clothes, dishes and so forth. Laundering is in general performed by agitating the fabrics in a detergent solution for a certain period of time followed by rinsing the fabrics in water. Laundering may be carried out by hand or by using an automatic washing machine in domestic household or in industrial scale.

Much progress has been made in the last decades in developing detergent compositions that remove soil and stains from laundry items, however there is still a need for new detergent compositions with improved wash performance.

### SUMMARY OF THE INVENTION

The present invention concerns a detergent composition comprising a detergent adjunct ingredient and two or more enzymes selected from the group consisting of a) protease and alpha-amylase, b) protease and mannanase, c) mannanase and alpha-amylase and d) protease, mannanase and alpha-amylase.

The invention further concerns the use of two or more enzymes for releasing or removing soil or stains from an item, wherein the enzymes are selected from the group consisting of a) protease and alpha-amylase, b) protease and mannanase, c) mannanase and alpha-amylase and d) protease, mannanase and alpha-amylase.

Further, the invention concerns a cleaning or laundering method, for cleaning or laundering an item comprising the steps of: 1) Exposing the item to a wash liquor comprising a detergent composition according to the invention or exposing the item to two or more enzymes selected from the group consisting of a) protease and alpha-amylase, b) protease and mannanase, c) mannanase and alpha-amylase and d) protease, mannanase and alpha-amylase, 2) completing at least one wash cycle; and 3) optionally rinsing the item, wherein the item is a textile, a fabric, a dishware or a hard surface.

### Definitions

A-, B- and C-domains: The structure of alpha-amylases comprises three distinct domains A, B and C, see, e.g., Machius et al., 1995, J. Mol. Biol. 246: 545-559. The term “domain” means a region of a polypeptide that in itself forms a distinct and independent substructure of the whole molecule. Alpha-amylases consist of a beta/alpha-8 barrel harboring the active site residues, which is denoted the A-domain, a rather long loop between the beta-sheet 3 and alpha-helix 3, which is denoted the B-domain (together; “A and B domain”), and a C-domain and in some cases also a carbohydrate binding domain (e.g., WO 2005/001064; Machius et al., supra).

The domains of an alpha-amylase can be determined by structure analysis such as using crystallographically techniques. An alternative method for determining the domains of an alpha-amylase is by sequence alignment of the amino acid sequence of the alpha-amylase with another alpha-amylase for which the domains have been determined. The sequence that aligns with, e.g., the C-domain sequence in the alpha-amylase for which the C-domain has been determined can be considered the C-domain for the given alpha-amylase.

A and B domain: The term “A and B domain” as used herein means these two domains taken as one unit, whereas the C domain is another unit of the alpha-amylases. Thus, the amimo acid sequence of the “A and B domain” is understood as one sequence or one part of a sequence of an alpha-amylase comprising an “A and B domain” and other domains (such as the C domain). As used herein, the “A and B domain” of an alpha-amylase corresponds to amino acids 1-399 of SEQ ID NO: 7.

Alpha-amylase: The term “alpha-amylase” is synonymous with the term “polypeptides having alpha-amylase activity”. “Alpha-amylase activity” means the activity of alpha-1,4-glucan-4-glucanohydrolases, E.C. 3.2.1.1, which constitute a group of enzymes, catalyzing hydrolysis of starch and other linear and branched 1,4-glucosidic oligo- and polysaccharides. For purposes of the present invention, alpha-amylase activity is determined according to the procedure described in the Methods. In one aspect, the alpha-amylases of the present invention have at least 20%, e.g., at least 40%, at least 50%, at least 60%, at least 70%, at least 80%, at least 90%, at least 95% or at least 100% of the alpha-amylase activity of the mature polypeptide of SEQ ID NO: 18.

The term automatic dishwashing detergent composition refers to compositions comprising detergent components, which composition is intended for cleaning dishware such as plates, cups, glasses, bowls, cutlery such as spoons, knives, forks, serving utensils, ceramics, plastics, metals, china, glass and acrylics in a dishwashing machine. The terms encompass any materials/compounds selected for domestic or industrial washing applications and the form of the product can be liquid, powder or granulate. In addition to lipase, the automatic dishwashing detergent composition contains detergent components such as polymers, bleaching systems, bleach activators, bleach catalysts, silicates, dyestuff and metal care agents.

C domain: As used herein, the “C domain” of an alpha-amylase corresponds to amino acids 400-485 of SEQ ID NO: 9. Thus, the C domain of an alpha amylase may be found by alignment of said alpha amylase with the alpha amylase of SEQ ID NO: 1. The part of said alpha amylase that aligns with amino acids 400-485 of SEQ ID NO: 9 is according to the present invention “the C domain” of the alpha amylase.

Complex stain means a stain composed of different types of soil, e.g. food stains comprising various raw materials such as protein(s) and/or proteinaceous substances, starch(es) or starch-like substances, oily and/or lipid substances, and polysaccharide texture modifying substances. The complex stains could possibly but not necessarily be in combination with dye substances and/or particulate substances such as particles with a heterogeneous nature with varying surface energies. These substances may found either in between textile fibers and microfibers of a fabric (natural or synthetic) and/or in a layer on the surface of the fabric. Non-exclusive examples of such stains could be salad dressings, puddings, sauces, gravies, ice creams, mayonnaises, marmalades, yogurts, and other processed or homemade foods with or without dye substances and/or particulate substances added.

Detergent adjunct ingredient: The detergent adjunct ingredient is different to the at least two enzymes of this invention. The precise nature of these additional adjunct components, and levels of incorporation thereof, will depend on the physical form of the composition and the nature of the operation for which it is to be used. Suitable adjunct materials include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Detergent components: the term “detergent components” is defined herein to mean the types of chemicals which can be used in detergent compositions for automatic dishwashing. Examples of detergent components are polymers, bleaching systems, bleach activators, bleach catalysts, silicates, dyestuff and metal care agents.

Detergent composition: the term “detergent composition” refers to compositions that find use in the removal of undesired compounds from items to be cleaned, such as textiles, dishes, and hard surfaces. The detergent composition may be used to e.g. clean textiles, dishes and hard surfaces for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish wash detergents). In addition to containing a bifunctional compound of the invention, the detergent formulation may contain one or more additional enzymes (such as proteases, alpha-amylases, lipases, cutinases, cellulases, endoglucanases, xyloglucanases, pectinases, pectin lyases, xanthanases, peroxidaes, haloperoxygenases, catalases and mannanases, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Dishware: The term dish ware is intended to mean any form of kitchen utensil, dinner set or tableware such as but not limited to pans, plates, cops, knives, forks, spoons, porcelain etc.

Dish washing composition: The term “dish washing composition” refers to compostions intended for cleaning dishes, table ware, pots, pans, cutlery and all forms of compositions for cleaning hard surfaces areas in kitchens. The present invention is not restricted to any particular type of dish wash composition or any particular detergent.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides.

Expression: The term “expression” includes any step involved in the production of a variant including, but not limited to, transcription, post-transcriptional modification, translation, post-translational modification, and secretion.

Fragment: The term “fragment” means a polypeptide having one or more (e.g., several) amino acids absent from the amino and/or carboxyl terminus of a mature polypeptide; wherein the fragment has alpha-amylase activity.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.

Improved wash performance: The term “improved wash performance” is defined herein as enzymes displaying an increased wash performance relative to the wash performance of similar item without the enzymes present e.g. by increased stain removal. The term “improved wash performance” includes wash performance in laundry but also e.g. in hard surface cleaning such as automated dish wash (ADW).

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 483 of SEQ ID NO: 18. In another aspect, the mature polypeptide is comprised of amino acids 1-399 of SEQ ID NO 7 and amino acids 398-483 of SEQ ID NO: 9. In yet another aspect, the mature polypeptide is a variant of amino acids 1-399 of SEQ ID NO 7 and amino acids 398-483 of SEQ ID NO: 9 having a deletion of any two amino acids of amino acids 181, 182, 183 and 184, such as amino acids 181 and 182 or 183 and 184 of SEQ ID NO:1. Such variants may further comprise substitutions e.g. in positions 403, 419, 420 and/or 426 of SEQ ID NO: 9. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mutant: The term “mutant” means a polynucleotide encoding a variant.

ΔRem enzyme value: The term “Delta enzyme remission value” is defined herein as the result of a reflectance or remission measurement at 460 nm. The swatch is measured with one swatch of similar colour as background, preferably a swatch from a repetition wash. A swatch representing each swatch type is measured before wash. The Delta enzyme remission is the remission value of the swatch washed in detergent with the at least two enzyme present minus the remission value of a similar swatch washed in a detergent without enzyme present.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.

Variant: The term “variant” means a polypeptide having alpha-amylase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. The variants of the present invention have at least 20%, e.g., at least 40%, at least 50%, at least 60%, at least 70%, at least 80%, at least 90%, at least 95% or at least 100% of the alpha-amylase activity of the mature polypeptide of SEQ ID NO: 18.

Wash cycle: The term “wash cycle” is defined herein as a washing operation wherein textiles are immersed in the wash liquor, mechanical action of some kind is applied to the textile in order to release stains and to facilitate flow of wash liquor in and out of the textile and finally the superfluous wash liquor is removed. After one or more wash cycles, the textile is generally rinsed and dried.

Wash liquor: The term “wash liquor” is defined herein as the solution or mixture of water and detergents optionally including the multi enzyme composition used for laundering textiles.

Wash performance: The term “wash performance” is used as an enzyme's ability to remove stains present on the object to be cleaned during e.g. wash or hard surface cleaning. The improvement in the wash performance may be quantified by measuring the Rem enzyme value. See also the wash performance test in Example 2 herein. The term “wash performance” and “dish wash performance” are used interchangeably.

Whiteness: The term “Whiteness” is defined herein as a broad term with different meanings in different regions and for different consumers. Loss of whiteness can e.g. be due to greying, yellowing, or removal of optical brighteners/hueing agents. Greying and yellowing can be due to soil redeposition, body soils, colouring from e.g. iron and copper ions or dye transfer. Whiteness might include one or several issues from the list below: colourant or dye effects; incomplete stain removal (e.g. body soils, sebum etc.); redeposition (greying, yellowing or other discolourations of the object) (removed soils reassociate with other parts of textile, soiled or unsoiled); chemical changes in textile during application; and clarification or brightening of colours.

Conventions for Designation of Variants

For purposes of the present invention, unless otherwise stated, the mature polypeptide disclosed in SEQ ID NO: 18 is used to determine the corresponding amino acid residue in another alpha-amylase. The amino acid sequence of another alpha-amylase is aligned with the mature polypeptide disclosed in SEQ ID NO: 18, and based on the alignment, the amino acid position number corresponding to any amino acid residue in the mature polypeptide disclosed in SEQ ID NO: 18 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another alpha-amylase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the mature polypeptide of SEQ ID NO: 18 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. In situations where the amino acid at a given position may be substituted for any other amino acid it is designated T226ACDEFGHIKLMNPQRSWVY. Accordingly, this means that threonine at position 226 may be substituted with one amino acid selected from the group of A, C, D, E, F, G, H, I, K, L, M, N, P, Q, R, S, W, V or Y. Likewise, in situations where the amino acid at a given position may be substituted for one amino acid selected from a specific group of amino acids, e.g. where the threonine at position 226 may be substituted with any of tyrosine, phenylalanine or histidine it is designated T226YFH. The different alterations at a given position may also be separated by a comma, e.g., “Arg170Tyr,Glu” or “R170Y,E” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

### DETAILED DESCRIPTION OF THE INVENTION

The present invention concerns a detergent composition comprising a detergent adjunct ingredient and two or more enzymes selected from the group consisting of
a. protease and alpha-amylase,
b. protease and mannanase,
c. mannanase and alpha-amylase; and
d. protease, mannanase and alpha-amylase.

The inventor has surprisingly found that using the above-mentioned combination of protease, alpha-amylase and mannanase shows increased wash performance. The mentioned combination of enzymes has shown to increase the release and removal of soil and stains from items. The item can be a textile, a fabric, a hard surface or a dishware. The exact combination of enzymes has a synergistic effect on the release or removal of stains from an item.

Removal of soil and stains from items such as laundry item can be quite difficult. Some stains are complex and difficult to remove, such as e.g. food stains comprising various types of soil, such as e.g. food stains comprising various raw materials such as protein(s) and/or proteinaceous substances, starch(es) or starch-like substances, oily and/or lipid substances, and polysaccharide texture modifying substances. These substances may be found either in between textile fibers and microfibers of a fabric (natural or synthetic) and/or in a layer on the surface of the fabric, which makes them difficult to remove. Commercial detergent compositions, which may also include enzymes, do not sufficiently release or remove complex stains. Also, in industrial and institutional washing complex stains can be difficult to remove. The inventor has found that by using a combination of a) protease and alpha-amylase, b) protease and mannanase, c) mannanase and alpha-amylase and d) protease, mannanase and alpha-amylase can solve this problem as these enzymes works synergistic on stains such as complex stains. The stains may then be removed easier e.g. without soaking or pre-spotting.

The protease, the alpha-amylase and the mannanase may be of animal, vegetable or microbial origin. In one embodiment the protease, the alpha-amylase and the mannanase is chemically modified or protein engineered.

In one embodiment of the invention, the alpha-amylase can be selected from the group consisting of:
a. a polypeptide having at least 90%, such as at least 95% sequence identity to SEQ ID NO: 1;
b. a polypeptide having at least 90%, such as at least 95% sequence identity to SEQ ID NO: 1 or a variant thereof wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444 when using SEQ ID NO: 1 for numbering;
c. a polypeptide having at least 90%, such as at least 95% sequence identity to SEQ ID NO: 2
d. a polypeptide having at least 90%, such as at least 95% sequence identity to the hybrid polypeptide of SEQ ID NO: 3
e. a polypeptide having at least 90%, such as at least 95% sequence identity to the hybrid polypeptide of SEQ ID NO: 3 wherein the hybrid polypeptide comprises a substitution, a deletion or an insertion in one of more of positions: 48, 49, 107, 156, 181, 190, 197, 201, 209 and/or 264 when using SEQ ID NO: 3 for numbering;
f. a polypeptide having at least 90%, such as at least 95% sequence identity to SEQ ID NO: 4;
g. a polypeptide having at least 90%, such as at least 95% sequence identity to SEQ ID NO: 4 or a variant thereof wherein the polypeptide comprises a substitution, a deletion or an insertion in one of more of positions: 181, 182, 183, 184, 195, 206, 212, 216 and/or 269 when using SEQ ID NO: 4 for numbering;
h. a polypeptide having at least 90%, such as at least 95% sequence identity to SEQ ID NO: 5, SEQ ID NO: 6 or SEQ ID NO: 7;
i. a polypeptide having at least 90%, such as at least 95% sequence identity to SEQ ID NO: 5, SEQ ID NO: 6 or SEQ ID NO: 7 or a variant thereof wherein the polypeptide comprises a substitution, a deletion or an insertion in one of more of positions: 140, 183, 184 195, 206, 243, 260, 304 and/or 476 when using SEQ ID NO: 5, SEQ ID NO: 6 or SEQ ID NO: 7 for numbering;
j. a polypeptide having at least 90%, such as at least 95% sequence identity to SEQ ID NO: 8;
k. a polypeptide having at least 90%, such as at least 95% sequence identity to SEQ ID NO: 9;
l. a polypeptide having at least 90%, such as at least 95% sequence identity to SEQ ID NO: 9 or a variant thereof wherein the polypeptide comprises a substitution, a deletion or an insertion in one of more of positions: 176, 177, 178, 179, 190, 201, 207, 211 and/or 264 when using SEQ ID NO: 9 for numbering;
m. a polypeptide having at least 90%, such as at least 95% sequence identity to SEQ ID NO: 10;
n. a polypeptide having at least 90%, such as at least 95% sequence identity to SEQ ID NO: 10 or a variant thereof wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475 when using SEQ ID NO: 10 for numbering;
o. a polypeptide having at least 90%, such as at least 95% sequence identity to SEQ ID NO: 11;
p. a polypeptide having at least 90%, such as at least 95% sequence identity to SEQ ID NO: 11 or a variant thereof wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484 when using SEQ ID NO: 11 for numbering;
q. a polypeptide having at least 80%, at least 85%, at least 90%, such as at least 95% sequence identity to SEQ ID NO: 18;
r. a polypeptide having at least 80%, at least 85%, at least 90%, such as at least 95% sequence identity to SEQ ID NO: 20;
s. a polypeptide having at least 80%, at least 85%, at least 90%, such as at least 95% sequence identity to SEQ ID NO: 21; and
t. a polypeptide having at least 80%, at least 85%, at least 90%, such as at least 95% sequence identity to SEQ ID NO: 22.

The protease can be a serine protease or a metalloprotease, preferably an alkaline microbial protease or a trypsin-like protease. The protease is selected from the group consisting of Bacillus, e.g., subtilisin Novo, subtilisin Carlsberg, subtilisin 309, subtilisin 147, subtilisin 168, trypsin of bovine origin, trypsin of porcine origin and Fusarium protease.

In one embodiment of the invention, the protease is selected from the group consisting of:
a. a polypeptide having at least 90%, such as at least 95% sequence identity to SEQ ID NO: 12;
b. a polypeptide having at least 90%, such as at least 95% sequence identity to SEQ ID NO: 12 or a variant thereof wherein the polypeptide comprises a substitution in one or more of positions: 9, 15, 27, 36, 68, 76, 87, 95, 96, 97, 98, 99, 100, 101, 102, 103, 104, 106, 118, 120, 123, 128, 129, 130, 160, 167, 170, 194, 195, 218, 222, 232, 235, 236, 245, 248, 252 and/or 274 using BPN′ numbering;
c. a polypeptide having at least 90%, such as at least 95% sequence identity to SEQ ID NO: 13; and
d. a polypeptide having at least 90%, such as at least 95% sequence identity SEQ ID NO: 13 or a variant thereof wherein the polypeptide comprises a substitution in one or more of positions: 3, 4, 99, 101, 103, 104, 159, 194, 199, 205 and/or 217 when using SEQ ID NO: 13 for numbering.

The protease can be a polypeptide having at least 90%, such as at least 95% sequence identity to SEQ ID NO: 12 comprises the following modifications: Y167A+R170S+A194P.

In one embodiment, the protease is SEQ ID NO: 12 having the modifications: Y167A+R170S+A194P.

In one embodiment of the invention, the mannanase is selected from the group consisting of:
a. a polypeptide having at least 80%, such as at least 85%, at least 90%, or at least 95% sequence identity to amino acids 31 to 330 of SEQ ID NO: 14;
b. a polypeptide having at least 80%, such as at least 85%, at least 90%, or at least 95% sequence identity to amino acids 32 to 334 of SEQ ID NO: 15;
c. a polypeptide having at least 80%, such as at least 85%, at least 90%, or at least 95% sequence identity to amino acids 33 to 331 of SEQ ID NO: 16; and
d. a polypeptide having at least 80%, such as at least 85%, at least 90%, or at least 95% sequence identity to amino acids 68 to 369 of SEQ ID NO: 17.

The various proteases, alpha-amylases and mannanases can be combined in different ways so that the combination of enzymes shows a synergistic effect. In one embodiment of the invention the at least two enzymes can be selected from the group consisting of:
a. An alpha-amylase having at least 90%, such as at least 95% sequence identity to SEQ ID NO: 9 or SEQ ID NO: 11 and a protease having at least 90%, such as at least 95% sequence identity to SEQ ID NO: 12;
b. An alpha-amylase having at least 90%, such as at least 95% sequence identity to SEQ ID NO: 9 or SEQ ID NO: 11; a protease having at least 90%, such as at least 95% sequence identity to SEQ ID NO: 12 and a mannanase having at least 80%, such as at least 85%, at least 90%, or at least 95% sequence identity to amino acids 31 to 330 of SEQ ID NO: 14;
c. An alpha-amylase having at least 90%, such as at least 95% sequence identity to SEQ ID NO: 9 or SEQ ID NO: 11 and a mannanase having at least 80%, such as at least 85%, at least 90%, or at least 95% sequence identity to amino acids 31 to 330 of SEQ ID NO: 14;
d. An alpha-amylase having at least 90%, such as at least 95% sequence identity to SEQ ID NO: 21 or SEQ ID NO: 22 and a protease having at least 90%, such as at least 95% sequence identity to SEQ ID NO: 12;
e. An alpha-amylase having at least 90%, such as at least 95% sequence identity to SEQ ID NO: 21 or SEQ ID NO: 22; a protease having at least 90%, such as at least 95% sequence identity to SEQ ID NO: 12 and a mannanase having at least 80%, such as at least 85%, at least 90%, or at least 95% sequence identity to amino acids 31 to 330 of SEQ ID NO: 14;
f. An alpha-amylase having at least 90%, such as at least 95% sequence identity to SEQ ID NO: 21 or SEQ ID NO: 22 and a mannanase having at least 80%, such as at least 85%, at least 90%, or at least 95% sequence identity to amino acids 31 to 330 of SEQ ID NO: 14;
g. a protease having at least 90%, such as at least 95% sequence identity to SEQ ID NO: 12 and a mannanase having at least 80%, such as at least 85%, at least 90%, or at least 95% sequence identity to amino acids 31 to 330 of SEQ ID NO: 14;
h. a polypeptide having at least 80%, at least 85%, at least 90%, such as at least 95% sequence identity to SEQ ID NO: 18 and a protease having at least 90%, such as at least 95% sequence identity to SEQ ID NO: 12;
i. a polypeptide having at least 80%, at least 85%, at least 90%, such as at least 95% sequence identity to SEQ ID NO: 18, a protease having at least 90%, such as at least 95% sequence identity to SEQ ID NO: 12; a mannanase having at least 80%, such as at least 85%, at least 90%, or at least 95% sequence identity to amino acids 31 to 330 of SEQ ID NO: 14;
j. a polypeptide having at least 80%, at least 85%, at least 90%, such as at least 95% sequence identity to SEQ ID NO: 18 and a mannanase having at least 80%, such as at least 85%, at least 90%, or at least 95% sequence identity to amino acids 31 to 330 of SEQ ID NO: 14;
k. a polypeptide having at least 80%, at least 85%, at least 90%, such as at least 95% sequence identity to SEQ ID NO: 18 and a protease having at least 90%, such as at least 95% sequence identity to SEQ ID NO: 12 comprising the following modifications: Y167A+R170S+A194P.
l. a polypeptide having at least 80%, at least 85%, at least 90%, such as at least 95% sequence identity to SEQ ID NO: 18, a protease having at least 90%, such as at least 95% sequence identity to SEQ ID NO: 12 comprising the following modifications: Y167A+R170S+A194P, and a mannanase having at least 80%, such as at least 85%, at least 90%, or at least 95% sequence identity to amino acids 31 to 330 of SEQ ID NO: 14; and
m. a protease having at least 90%, such as at least 95% sequence identity to SEQ ID NO: 12 comprising the following modifications: Y167A+R170S+A194P and a mannanase having at least 80%, such as at least 85%, at least 90%, or at least 95% sequence identity to amino acids 31 to 330 of SEQ ID NO: 14;

The concentration of the enzymes may be in the range of 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The detergent composition according to the invention comprises a detergent adjunct ingredient in addition to the enzymes showing synergy. The detergent adjunct ingredient can be selected from the group consisting of surfactants, builders, flocculating aid, chelating agents, dye transfer inhibitors, enzymes, enzyme stabilizers, enzyme inhinitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

In one embodiment of the invention, the detergent composition further comprises one or more enzymes selected from the group consisting of proteases, lipases, cutinases, alpha-amylases, carbohydrases, cellulases, pectinases, mannanases, arabinases, galactanases, xylanases, peroxidases and oxidases.

The detergent composition can be formulated as a bar, a homogenous tablet, a tablet having two or more layers, a pouch having one or more compartments, a regular or compact powder, a granule, a paste, a gel, or a regular, compact or concentrated liquid. In one embodiment, the detergent composition is a liquid detergent, a powder detergent or a granule detergent. In one embodiment the detergent composition is a liquid detergent and the detergent composition may then comprise an enzyme inhibitor, such as the enzyme inhibitors described in EP 2271660.

In one embodiment, the detergent composition comprises a surfactant and an alpha-amylase having at least 90%, such as at least 95%, sequence identity to SEQ ID NO: 9 or SEQ ID NO: 11 and a protease having at least 90%, such as at least 95%, sequence identity to SEQ ID NO: 12 and is used for removing or releasing soil or stains from a textile, fabric or dishware.

In one embodiment, the detergent composition comprises a surfactant and an alpha-amylase having at least 90% sequence identity to SEQ ID NO: 9 or SEQ ID NO: 11; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90%, such as at least 95% sequence identity to SEQ ID NO: 9 or SEQ ID NO: 11 and a mannanase having at least 80%, such as at least 85%, at least 90%, or at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22 and a protease having at least 90% sequence identity to SEQ ID NO: 12 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22 and a protease having at least 95% sequence identity to SEQ ID NO: 12 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22 and a protease having at least 95% sequence identity to SEQ ID NO: 12 and is used for removing or releasing soil or stains from a textile, fabric or dishware.

In one embodiment the detergent composition comprises a surfactant and an alpha-amylase having at least 95% sequence identity to SEQ ID NO: 21 or SEQ ID NO: 22 and a protease having at least 90% sequence identity to SEQ ID NO: 12 and is used for removing or releasing soil or stains from a textile, fabric or dishware.

In one embodiment, the detergent composition comprises a surfactant and an alpha-amylase having at least 100 sequence identity to SEQ ID NO: 21 or SEQ ID NO: 22 and a protease having at least 90% sequence identity to SEQ ID NO: 12 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22 and a protease having at least 95% sequence identity to SEQ ID NO: 12 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22 and a protease having 100% sequence identity to SEQ ID NO: 12 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22 and a protease having 100% sequence identity to SEQ ID NO: 12 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22 and a protease having 100% sequence identity to SEQ ID NO: 12 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22; a protease having at least 95% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22; a protease having 100% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22; a protease having at least 95% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22; a protease having at least 95% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22; a protease having at least 95% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22; a protease having at least 95% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 90% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 90% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 90% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 90% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 95% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 95% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 95% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 95% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having at least 95% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having 100% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having 100% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having 100% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having 100% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22; a protease having 100% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 90% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 90% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 90% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 90% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 95% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 95% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 95% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 95% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 95% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having 100% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having 100% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having 100% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having 100% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having 100% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 21 or SEQ ID NO: 22 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 21 or SEQ ID NO: 22 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22 and a mannanase having at least 100% sequence identity to amino acids 31 to 330 of SEQ ID NO: 14 and is used for removing or releasing soil or stains from a textile, fabric or dishware.

In one embodiment, the detergent composition comprises a surfactant and a protease having at least 90% sequence identity to SEQ ID NO: 12 and a mannanase having at least 80% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 90% sequence identity to SEQ ID NO: 12 and a mannanase having at least 85% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 90% sequence identity to SEQ ID NO: 12 and a mannanase having at least 90% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 90% sequence identity to SEQ ID NO: 12 and a mannanase having at least 95% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 90% sequence identity to SEQ ID NO: 12 and a mannanase having 100% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 95% sequence identity to SEQ ID NO: 12 and a mannanase having at least 85% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 95% sequence identity to SEQ ID NO: 12 and a mannanase having at least 80% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 95% sequence identity to SEQ ID NO: 12 and a mannanase having at least 90% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 95% sequence identity to SEQ ID NO: 12 and a mannanase having at least 95% sequence identity to amino acids 31 to 330 of SEQ ID NO: 14 and may be used for removing or releasing soil or stains from a textile, fabric or dishware.

In one embodiment, the detergent composition comprises a surfactant and a protease having at least 95% sequence identity to SEQ ID NO: 12 and a mannanase having 100% sequence identity to amino acids 31 to 330 of SEQ ID NO: 14 and may be used for removing or releasing soil or stains from a textile, fabric or dishware.

In one embodiment, the detergent composition comprises a surfactant and a protease having 100% sequence identity to SEQ ID NO: 12 and a mannanase having at least 80% sequence identity to amino acids 31 to 330 of SEQ ID NO: 14 and may be used for removing or releasing soil or stains from a textile, fabric or dishware.

In one embodiment, the detergent composition comprises a surfactant and a protease having 100% sequence identity to SEQ ID NO: 12 and a mannanase having at least 85% sequence identity to amino acids 31 to 330 of SEQ ID NO: 14 and may be used for removing or releasing soil or stains from a textile, fabric or dishware.

In one embodiment, the detergent composition comprises a surfactant and a protease having 100% sequence identity to SEQ ID NO: 12 and a mannanase having at least 90% sequence identity to amino acids 31 to 330 of SEQ ID NO: 14 and may be used for removing or releasing soil or stains from a textile, fabric or dishware.

In one embodiment, the detergent composition comprises a surfactant and a protease having 100% sequence identity to SEQ ID NO: 12 and a mannanase having at least 95% sequence identity to amino acids 31 to 330 of SEQ ID NO: 14 and may be used for removing or releasing soil or stains from a textile, fabric or dishware.

In one embodiment, the detergent composition comprises a surfactant and a protease having 100% sequence identity to SEQ ID NO: 12 and a mannanase having at least 100% sequence identity to amino acids 31 to 330 of SEQ ID NO: 14 and may be used for removing or releasing soil or stains from a textile, fabric or dishware.

In one embodiment, the detergent composition comprises a surfactant and a polypeptide having at least 80% sequence identity to SEQ ID NO: 18 and a protease having at least 90% sequence identity to SEQ ID NO: 12 and may be used for removing or releasing soil or stains from a textile, fabric or dishware.

In one embodiment, the detergent composition comprises a surfactant and a polypeptide having at least 80% sequence identity to SEQ ID NO: 18 and a protease having at least 95% sequence identity to SEQ ID NO: 12 and may be used for removing or releasing soil or stains from a textile, fabric or dishware.

In one embodiment, the detergent composition comprises a surfactant and a polypeptide having at least 80% sequence identity to SEQ ID NO: 18 and a protease having 100% sequence identity to SEQ ID NO: 12 and may be used for removing or releasing soil or stains from a textile, fabric or dishware.

In one embodiment, the detergent composition comprises a surfactant and a polypeptide having at least 85% sequence identity to SEQ ID NO: 18 and a protease having at least 90% sequence identity to SEQ ID NO: 12 and may be used for removing or releasing soil or stains from a textile, fabric or dishware.

In one embodiment, the detergent composition comprises a surfactant and a polypeptide having at least 85% sequence identity to SEQ ID NO: 18 and a protease having at least 95% sequence identity to SEQ ID NO: 12 and may be used for removing or releasing soil or stains from a textile, fabric or dishware.

In one embodiment, the detergent composition comprises a surfactant and a polypeptide having at least 85% sequence identity to SEQ ID NO: 18 and a protease having 100% sequence identity to SEQ ID NO: 12 and may be used for removing or releasing soil or stains from a textile, fabric or dishware.

In one embodiment, the detergent composition comprises a surfactant and a polypeptide having at least 90% sequence identity to SEQ ID NO: 18 and a protease having at least 90% sequence identity to SEQ ID NO: 12 and may be used for removing or releasing soil or stains from a textile, fabric or dishware.

In one embodiment, the detergent composition comprises a surfactant and a polypeptide having at least 90% sequence identity to SEQ ID NO: 18 and a protease having at least 95% sequence identity to SEQ ID NO: 12 and may be used for removing or releasing soil or stains from a textile, fabric or dishware.

In one embodiment, the detergent composition comprises a surfactant and a polypeptide having at least 90% sequence identity to SEQ ID NO: 18 and a protease having 100% sequence identity to SEQ ID NO: 12 and may be used for removing or releasing soil or stains from a textile, fabric or dishware.

In one embodiment, the detergent composition comprises a surfactant and a polypeptide having at least 95% sequence identity to SEQ ID NO: 18 and a protease having at least 90% sequence identity to SEQ ID NO: 12 and may be used for removing or releasing soil or stains from a textile, fabric or dishware.

In one embodiment, the detergent composition comprises a surfactant and a polypeptide having at least 95% sequence identity to SEQ ID NO: 18 and a protease having at least 95% sequence identity to SEQ ID NO: 12 and may be used for removing or releasing soil or stains from a textile, fabric or dishware.

In one embodiment, the detergent composition comprises a surfactant and a polypeptide having at least 95% sequence identity to SEQ ID NO: 18 and a protease having 100% sequence identity to SEQ ID NO: 12 and may be used for removing or releasing soil or stains from a textile, fabric or dishware.

In one embodiment, the detergent composition comprises a surfactant and a polypeptide having 100% sequence identity to SEQ ID NO: 18 and a protease having at least 90% sequence identity to SEQ ID NO: 12 and may be used for removing or releasing soil or stains from a textile, fabric or dishware.

In one embodiment, the detergent composition comprises a surfactant and a polypeptide having 100% sequence identity to SEQ ID NO: 18 and a protease having at least 95% sequence identity to SEQ ID NO: 12 and may be used for removing or releasing soil or stains from a textile, fabric or dishware.

In one embodiment, the detergent composition comprises a surfactant and a polypeptide having 100% sequence identity to SEQ ID NO: 18 and a protease having 100% sequence identity to SEQ ID NO: 12 and may be used for removing or releasing soil or stains from a textile, fabric or dishware.

In one embodiment, the detergent composition comprises a surfactant and a polypeptide having at least 80% sequence identity to SEQ ID NO: 18, a protease having at least 90% sequence identity to SEQ ID NO: 12; a mannanase having at least 80% sequence identity to amino acids 31 to 330 of SEQ ID NO: 14 and may be used for removing or releasing soil or stains from a textile, fabric or dishware.

In one embodiment, the detergent composition comprises a surfactant and an alpha-amylase having at least 80% sequence identity to SEQ ID NO: 18; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 90% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 90% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 90% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 90% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 95% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 95% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 95% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 95% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 80% sequence identity to SEQ ID NO: 18; a protease having at least 95% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 90% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 90% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 90% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 90% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 95% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 95% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 95% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 95% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 85% sequence identity to SEQ ID NO: 18; a protease having at least 95% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 18; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 18; a protease having at least 95% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 18; a protease having 100% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 18; a protease having at least 95% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 18; a protease having at least 95% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 18; a protease having at least 95% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 90% sequence identity to SEQ ID NO: 18; a protease having at least 95% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 90% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 90% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 90% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 90% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 95% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 95% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 95% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 95% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having at least 95% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having 100% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having 100% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having 100% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having 100% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having at least 95% sequence identity to SEQ ID NO: 18; a protease having 100% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 90% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 90% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 90% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 90% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 21 or SEQ ID NO: 22; a protease having at least 90% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 95% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 95% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 95% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 95% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having at least 95% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having 100% sequence identity to SEQ ID NO: 12 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having 100% sequence identity to SEQ ID NO: 12 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having 100% sequence identity to SEQ ID NO: 12 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having 100% sequence identity to SEQ ID NO: 12 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and an alpha-amylase having 100% sequence identity to SEQ ID NO: 18; a protease having 100% sequence identity to SEQ ID NO: 12 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 80% sequence identity to SEQ ID NO: 18 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 90% sequence identity to SEQ ID NO: 18 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 90% sequence identity to SEQ ID NO: 18 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 90% sequence identity to SEQ ID NO: 18 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 90% sequence identity to SEQ ID NO: 18 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 90% sequence identity to SEQ ID NO: 18 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 95% sequence identity to SEQ ID NO: 18 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 95% sequence identity to SEQ ID NO: 18 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 95% sequence identity to SEQ ID NO: 18 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 95% sequence identity to SEQ ID NO: 18 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 95% sequence identity to SEQ ID NO: 18 and a mannanase having 100% sequence identity to amino acids 31 to 330 of SEQ ID NO: 14 and is used for removing or releasing soil or stains from a textile, fabric or dishware.

In one embodiment, the detergent composition comprises a surfactant and a polypeptide having 100% sequence identity to SEQ ID NO: 18 and a mannanase having at least 80% sequence identity to amino acids 31 to 330 of SEQ ID NO: 14 and is used for removing or releasing soil or stains from a textile, fabric or dishware.

In one embodiment, the detergent composition comprises a surfactant and a polypeptide having 100% sequence identity to SEQ ID NO: 18 and a mannanase having at least 85% sequence identity to amino acids 31 to 330 of SEQ ID NO: 14 and is used for removing or releasing soil or stains from a textile, fabric or dishware.

In one embodiment, the detergent composition comprises a surfactant and a polypeptide having 100% sequence identity to SEQ ID NO: 18 and a mannanase having at least 90% sequence identity to amino acids 31 to 330 of SEQ ID NO: 14 and is used for removing or releasing soil or stains from a textile, fabric or dishware.

In one embodiment, the detergent composition comprises a surfactant and a polypeptide having 100% sequence identity to SEQ ID NO: 18 and a mannanase having at least 95% sequence identity to amino acids 31 to 330 of SEQ ID NO: 14 and is used for removing or releasing soil or stains from a textile, fabric or dishware.

In one embodiment, the detergent composition comprises a surfactant and a polypeptide having 100% sequence identity to SEQ ID NO: 18 and a mannanase having at least 100% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 80%, at least 85%, at least 90%, such as at least 95% sequence identity to SEQ ID NO: 18 and a protease having at least 90%, such as at least 95% sequence identity to SEQ ID NO: 12 comprising the following modifications: Y167A+R170S+A194P and is used for removing or releasing soil or stains from a textile, fabric or dishware.

In one embodiment, the detergent composition comprises a surfactant and a polypeptide having at least 80%, at least 85%, at least 90%, such as at least 95% sequence identity to SEQ ID NO: 18, a protease having at least 90%, such as at least 95% sequence identity to SEQ ID NO: 12 comprising the following modifications: Y167A+R170S+A194P, and a mannanase having at least 80%, such as at least 85%, at least 90%, or at least 95% sequence identity to amino acids 31 to 330 of SEQ ID NO: 14 and is used for removing or releasing soil or stains from a textile, fabric or dishware.

In one embodiment, the detergent composition comprises a surfactant and a protease having at least 90%, such as at least 95% sequence identity to SEQ ID NO: 12 comprising the following modifications: Y167A+R170S+A194P and a mannanase having at least 80%, such as at least 85%, at least 90%, or at least 95% sequence identity to amino acids 31 to 330 of SEQ ID NO: 14 and is used for removing or releasing soil or stains from a textile, fabric or dishware.

In one embodiment, the detergent composition comprises a surfactant and a polypeptide having at least 80%, at least 85%, at least 90%, such as at least 95% sequence identity to SEQ ID NO: 18 and a protease having at least 90%, such as at least 95% sequence identity to SEQ ID NO: 12 comprising the following modifications: Y161A+R164S+A188P and is used for removing or releasing soil or stains from a textile, fabric or dishware.

In one embodiment, the detergent composition comprises a surfactant and a polypeptide having at least 80%, at least 85%, at least 90%, such as at least 95% sequence identity to SEQ ID NO: 18, a protease having at least 90%, such as at least 95% sequence identity to SEQ ID NO: 12 comprising the following modifications: Y161A+R164S+A188P, and a mannanase having at least 80%, such as at least 85%, at least 90%, or at least 95% sequence identity to amino acids 31 to 330 of SEQ ID NO: 14 and is used for removing or releasing soil or stains from a textile, fabric or dishware.

In one embodiment, the detergent composition comprises a surfactant and a protease having at least 90%, such as at least 95% sequence identity to SEQ ID NO: 12 comprising the following modifications: Y161A+R164S+A188P and a mannanase having at least 80%, such as at least 85%, at least 90%, or at least 95% sequence identity to amino acids 31 to 330 of SEQ ID NO: 14 and is used for removing or releasing soil or stains from a textile, fabric or dishware.

The invention also concerns a cleaning or laundering method for cleaning or laundering an item comprising the steps of:
a. Exposing an item to a wash liquor comprising a detergent composition according to any of claims 1-16 or exposing the item to two or more enzymes selected from the group consisting of
   i. protease and alpha-amylase,
   ii. protease and mannanase,
   iii. mannanase and alpha-amylase and
   iv. protease, mannanase and alpha-amylase.
b. Completing at least one wash cycle; and
c. Optionally rinsing the item
   wherein the item is a textile, a fabric, a dishware or a hard surface.

The pH of the wash liquor may be in the range of 7 to 10.5. The pH of the wash liquor can be in the range of 7 to 9, in the range of 7 to 8 or in the range of 7 to 7.5.

In one embodiment of the invention, the temperature of the wash liquor may be in the range of 5° C. to 95° C., or in the range of 10° C. to 80° C., in the range of 10° C. to 70° C., in the range of 10° C. to 60° C., in the range of 10° C. to 50° C., in the range of 15° C. to 40° C., in the range of 20° C. to 30° C. or in the range of 10° C. to 15° C. In one embodiment the temperature of the wash liquor is 15° C.

When a wash cycle is completed the wash liquor can be drained. In one embodiment the wash liquor from a first wash cycle may be re-used in a second wash cycle. In one embodiment only part of the wash liquor is drained after completion of a wash cycle and the remaining wash liquor may be re-used in a second wash cycle and/or third wash cycle.

In one embodiment, the item is rinsed after being exposed to the wash liquor. For rinsing water or water comprising a conditioner may be used.

### Enzyme of the Present Invention

In one embodiment of the present invention, the a polypeptide of the present invention may be added to a detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The enzyme(s) of the detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708.

A polypeptide of the present invention may also be incorporated in the detergent formulations disclosed in WO97/07202, which is hereby incorporated by reference.

### Detergent Compositions

In one embodiment, the invention is directed to detergent compositions comprising an enzyme of the present invention in combination with one or more additional cleaning composition components. The choice of additional components is within the skill of the artisan and includes conventional ingredients, including the exemplary non-limiting components set forth below.

In one embodiment, the invention is directed to an ADW (Automatic Dish Wash) composition comprising an enzyme of the present invention in combination with one or more additional ADW composition components. The choice of additional components is within the skill of the artisan and includes conventional ingredients, including the exemplary non-limiting components set forth below.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 1% to about 40% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 1% to about 40% by weight of a zwitterionic surfactant. Non-limiting examples of zwitterionic surfactants include betaines such as alkyldimethylbetaines, sulfobetaines, and combinations thereof.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in laundry/ADW/hard surface cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Bleaching Systems

The detergent may contain 0-30% by weight, such as about 1% to about 20%, of a bleaching system. Any bleaching system known in the art for use in laundry/ADW/hard surface cleaning detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particulary preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

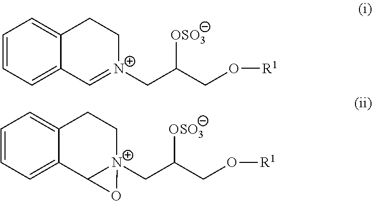

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) preformed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

### Anti-Foaming Agents

### Enzymes

The detergent additive as well as the detergent composition may comprise one or more additional enzymes such as a protease, lipase, cutinase, an alpha-amylase, carbohydrase, cellulase, pectinase, mannanase, arabinase, galactanase, xylanase, oxidase, e.g., a laccase, and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes NS), Whitezyme™ (Novozymes A/S), Clazinase™, and Puradax HA™ (Genencor International Inc.), and KAC-500(B)™ (Kao Corporation).

Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the 51 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes A/S), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases:

Suitable amylases which can be used in the present invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21D+D97N+V128I

wherein the variants optionally further comprises a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Excellase S, Preferenz S1000, Preferenz S100 and Preferenz S110 (from Genencor International Inc./DuPont).

Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry/ADW/hard surface cleaning detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry/ADW/hard surface cleaning detergents may be utilized. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2,2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, enzymes, optionally one or more additional enzymes, a protease inhibitor, and a salt of a monovalent cation and an organic anion may be prepared and the mixture is then plodded. The enzymes and optional additional enzymes may be added at the same time as the protease inhibitor for example in liquid form. Besides the mixing step and the plodding step, the process may further comprise the steps of milling, extruding, cutting, stamping, cooling and/or wrapping.

### Use in Detergents.

The polypeptides of the present invention may be added to and thus become a component of a detergent composition.

The detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the present invention provides a detergent additive comprising a polypeptide of the present invention as described herein.

The invention is further defined in the following paragraphs:
1. Detergent composition comprising a detergent adjunct ingredient and two or more enzymes selected from the group consisting of
   a. protease and alpha-amylase,
   b. protease and mannanase,
   c. mannanase and alpha-amylase and
   d. protease, mannanase and alpha-amylase.
2. Detergent composition according to paragraph 1, wherein the protease, the alpha-amylase and the mannanase is of animal, vegetable or microbial origin.
3. Detergent composition according to any of the preceding paragraphs, wherein the protease, the alpha-amylase and the mannanase is chemically modified or protein engineered.
4. Detergent composition according to any of the preceding paragraphs, wherein the alpha-amylase is of bacterial or fungal origin.
5. Detergent composition according to any of the preceding paragraphs, wherein the alpha-amylase is selected from the group consisting of:
   a. a polypeptide having at least 90%, such as at least 95% sequence identity to SEQ ID NO: 1;
   b. a polypeptide having at least 90%, such as at least 95% sequence identity to SEQ ID NO: 1 or a variant thereof wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444 when using SEQ ID NO: 1 for numbering;
   c. a polypeptide having at least 90%, such as at least 95% sequence identity to SEQ ID NO: 2
   d. a polypeptide having at least 90%, such as at least 95% sequence identity to the hybrid polypeptide of SEQ ID NO: 3
   e. a polypeptide having at least 90%, such as at least 95% sequence identity to the hybrid polypeptide of SEQ ID NO: 3 wherein the hybrid polypeptide comprises a substitution, a deletion or an insertion in one of more of positions: 48, 49, 107, 156, 181, 190, 197, 201, 209 and/or 264 when using SEQ ID NO: 3 for numbering;
   f. a polypeptide having at least 90%, such as at least 95% sequence identity to SEQ ID NO: 4;
   g. a polypeptide having at least 90%, such as at least 95% sequence identity to SEQ ID NO: 4 or a variant thereof wherein the polypeptide comprises a substitution, a deletion or an insertion in one of more of positions: 181, 182, 183, 184, 195, 206, 212, 216 and/or 269 when using SEQ ID NO: 4 for numbering;
   h. a polypeptide having at least 90%, such as at least 95% sequence identity to SEQ ID NO: 5, SEQ ID NO: 6 or SEQ ID NO: 7;
   i. a polypeptide having at least 90%, such as at least 95% sequence identity to SEQ ID NO: 5, SEQ ID NO: 6 or SEQ ID NO: 7 or a variant thereof wherein the polypeptide comprises a substitution, a deletion or an insertion in one of more of positions: 140, 183, 184 195, 206, 243, 260, 304 and/or 476 when using SEQ ID NO: 5, SEQ ID NO: 6 or SEQ ID NO: 7 for numbering;
   j. a polypeptide having at least 90%, such as at least 95% sequence identity to SEQ ID NO: 8;
   k. a polypeptide having at least 90%, such as at least 95% sequence identity to SEQ ID NO: 9;
   l. a polypeptide having at least 90%, such as at least 95% sequence identity to SEQ ID NO: 9 or a variant thereof wherein the polypeptide comprises a substitution, a deletion or an insertion in one of more of positions: 176, 177, 178, 179, 190, 201, 207, 211 and/or 264 when using SEQ ID NO: 9 for numbering;
   m. a polypeptide having at least 90%, such as at least 95% sequence identity to SEQ ID NO: 10;
   n. a polypeptide having at least 90%, such as at least 95% sequence identity to SEQ ID NO: 10 or a variant thereof wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475 when using SEQ ID NO: 10 for numbering;
   o. a polypeptide having at least 90%, such as at least 95% sequence identity to SEQ ID NO: 11;
   p. a polypeptide having at least 90%, such as at least 95% sequence identity to SEQ ID NO: 11 or a variant thereof wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484 when using SEQ ID NO: 11 for numbering; and
   q. a polypeptide having at least 80%, at least 85%, at least 90%, such as at least 95% sequence identity to SEQ ID NO: 18;
   r. a polypeptide having at least 80%, at least 85%, at least 90%, such as at least 95% sequence identity to SEQ ID NO: 20;
   s. a polypeptide having at least 80%, at least 85%, at least 90%, such as at least 95% sequence identity to SEQ ID NO: 21; and
6. a polypeptide having at least 80%, at least 85%, at least 90%, such as at least 95% sequence identity to SEQ ID NO: 22. Detergent composition to any of the preceding paragraphs, wherein the protease is a serine protease or a metalloprotease, preferably an alkaline microbial protease or a trypsin-like protease.
7. Detergent composition to any of the preceding paragraphs, wherein the protease is selected from the group consisting of Bacillus, e.g., subtilisin Novo, subtilisin Carlsberg, subtilisin 309, subtilisin 147, subtilisin 168, trypsin of bovine origin, trypsin of porcine origin and Fusarium protease.
8. Detergent composition according to any of the preceding paragraphs, wherein the protease is selected from the group consisting of:
   a. a polypeptide having at least 90%, such as at least 95% sequence identity to SEQ ID NO: 12;
   b. a polypeptide having at least 90%, such as at least 95% sequence identity to SEQ ID NO: 12 or a variant thereof wherein the polypeptide comprises a substitution in one or more of positions: 9, 15, 27, 36, 68, 76, 87, 95, 96, 97, 98, 99, 100, 101, 102, 103, 104, 106, 118, 120, 123, 128, 129, 130, 160, 167, 170, 194, 195, 218, 222, 232, 235, 236, 245, 248, 252 and/or 274 using BPN′ numbering;
   c. a polypeptide having at least 90%, such as at least 95% sequence identity to SEQ ID NO: 13; and
   d. a polypeptide having at least 90%, such as at least 95% sequence identity SEQ ID NO: 13 or a variant thereof wherein the polypeptide comprises a substitution in one or more of positions: 3, 4, 99, 101, 103, 104, 159, 194, 199, 205 and/or 217 when using SEQ ID NO: 13 for numbering.
9. Detergent composition according to paragraph 9, wherein the polypeptide having at least 90%, such as at least 95% sequence identity to SEQ ID NO: 12 comprises the following modifications: Y167A+R170S+A194P.
10. Detergent composition according to any of the preceding paragraphs, wherein the mannanase is selected from the group consisting of:
   a. a polypeptide having at least 80%, such as at least 85%, at least 90%, or at least 95% sequence identity to amino acids 31 to 330 of SEQ ID NO: 14;
   b. a polypeptide having at least 80%, such as at least 85%, at least 90%, or at least 95% sequence identity to amino acids 32 to 334 of SEQ ID NO: 15;
   c. a polypeptide having at least 80%, such as at least 85%, at least 90%, or at least 95% sequence identity to amino acids 33 to 331 of SEQ ID NO: 16; and
   d. a polypeptide having at least 80%, such as at least 85%, at least 90%, or at least 95% sequence identity to amino acids 68 to 369 of SEQ ID NO: 17.
11. Detergent composition according to any of the preceding paragraphs, comprising at least the following enzymes:
   a. An alpha-amylase having at least 90%, such as at least 95% sequence identity to SEQ ID NO: 9 or SEQ ID NO: 11 and a protease having at least 90%, such as at least 95% sequence identity to SEQ ID NO: 12;
   b. An alpha-amylase having at least 90%, such as at least 95% sequence identity to SEQ ID NO: 9 or SEQ ID NO: 11; a protease having at least 90%, such as at least 95% sequence identity to SEQ ID NO: 12 and a mannanase having at least 80%, such as at least 85%, at least 90% or at least 95% sequence identity to amino acids 31 to 330 of SEQ ID NO: 14;
   c. An alpha-amylase having at least 90%, such as at least 95% sequence identity to SEQ ID NO: 9 or SEQ ID NO: 11 and a mannanase having at least 80%, such as at least 85%, at least 90% or at least 95% sequence identity to amino acids 31 to 330 of SEQ ID NO: 14;
   d. An alpha-amylase having at least 90%, such as at least 95% sequence identity to SEQ ID NO: 21 or SEQ ID NO: 22 and a protease having at least 90%, such as at least 95% sequence identity to SEQ ID NO: 12;
   e. An alpha-amylase having at least 90%, such as at least 95% sequence identity to SEQ ID NO: 21 or SEQ ID NO: 22; a protease having at least 90%, such as at least 95% sequence identity to SEQ ID NO: 12 and a mannanase having at least 80%, such as at least 85%, at least 90% or at least 95% sequence identity to amino acids 31 to 330 of SEQ ID NO: 14;
   f. An alpha-amylase having at least 90%, such as at least 95% sequence identity to SEQ ID NO: 21 or SEQ ID NO: 22 and a mannanase having at least 80%, such as at least 85%, at least 90% or at least 95% sequence identity to amino acids 31 to 330 of SEQ ID NO: 14;
   g. a protease having at least 90%, such as at least 95% sequence identity to SEQ ID NO: 12 and a mannanase having at least 80%, such as at least 85%, at least 90% or at least 95% sequence identity to amino acids 31 to 330 of SEQ ID NO: 14;
   h. a polypeptide having at least 80%, at least 85%, at least 90%, such as at least 95% sequence identity to SEQ ID NO: 18 and a protease having at least 90%, such as at least 95% sequence identity to SEQ ID NO: 12;
   i. a polypeptide having at least 80%, at least 85%, at least 90%, such as at least 95% sequence identity to SEQ ID NO: 18, a protease having at least 90%, such as at least 95% sequence identity to SEQ ID NO: 12; a mannanase having at least 80%, such as at least 85%, at least 90% or at least 95% sequence identity to amino acids 31 to 330 of SEQ ID NO: 14;
   j. a polypeptide having at least 80%, at least 85%, at least 90%, such as at least 95% sequence identity to SEQ ID NO: 18 and a mannanase having at least 80%, such as at least 85%, at least 90% or at least 95% sequence identity to amino acids 31 to 330 of SEQ ID NO: 14;
   k. a polypeptide having at least 80%, at least 85%, at least 90%, such as at least 95% sequence identity to SEQ ID NO: 18 and a protease having at least 90%, such as at least 95% sequence identity to SEQ ID NO: 12 comprising the following modifications: Y167A+R170S+A194P.
   l. a polypeptide having at least 80%, at least 85%, at least 90%, such as at least 95% sequence identity to SEQ ID NO: 18, a protease having at least 90%, such as at least 95% sequence identity to SEQ ID NO: 12 comprising the following modifications: Y167A+R170S+A194P, and a mannanase having at least 80%, such as at least 85%, at least 90% or at least 95% sequence identity to amino acids 31 to 330 of SEQ ID NO: 14; or
   m. a protease having at least 90%, such as at least 95% sequence identity to SEQ ID NO: 12 comprising the following modifications: Y167A+R170S+A194P and a mannanase having at least 80%, such as at least 85%, at least 90% or at least 95% sequence identity to amino acids 31 to 330 of SEQ ID NO: 14;
12. Detergent composition according to any of the preceding paragraphs, wherein the detergent adjunct ingredient is selected from the group consisting of surfactants, builders, flocculating aid, chelating agents, dye transfer inhibitors, enzymes, enzyme stabilizers, enzyme inhinitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.
13. Detergent composition according to any of the preceding paragraphs, wherein the composition further comprises one or more enzymes selected from the group consisting of proteases, lipases, cutinases, alpha-amylases, carbohydrases, cellulases, pectinases, mannanases, arabinases, galactanases, xylanases, peroxidases and oxidases.
14. Detergent composition according to any of the preceding paragraphs, wherein the composition is a bar, a homogenous tablet, a tablet having two or more layers, a pouch having one or more compartments, a regular or compact powder, a granule, a paste, a gel, or a regular, compact or concentrated liquid.
15. Detergent composition according to any of the preceding paragraphs, wherein the composition is a liquid detergent, a powder detergent or a granule detergent.
16. Use of a combination of two or more enzymes for releasing or removing soil or stains from an item, wherein the enzymes are selected from the group consisting of
   a. protease and alpha-amylase,
   b. protease and mannanase,
   c. mannanase and alpha-amylase and
   d. protease, mannanase and alpha-amylase.
17. Use according to paragraph 16, wherein the stains are complex stains.
18. Use according to any of the preceding use paragraphs, wherein the wash performance of the two or more enzymes is synergistic.
19. Use according to any of the preceding use paragraphs, wherein the protease, the alpha-amylase and the mannanase is of animal, vegetable or microbial origin.
20. Use according to any of the preceding use paragraphs, wherein the protease, the alpha-amylase and the mannanase is chemically modified or protein engineered.
21. Use according to any of the preceding use paragraphs, wherein the alpha-amylase is of bacterial or fungal origin.
22. Use according to any of the preceding use paragraphs, wherein the alpha-amylase is selected from the group consisting of:
   a. a polypeptide having at least 90%, such as at least 95% sequence identity to SEQ ID NO: 1;
   b. a polypeptide having at least 90%, such as at least 95% sequence identity to SEQ ID NO: 1 or a variant thereof wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444 when using SEQ ID NO: 1 for numbering;
   c. a polypeptide having at least 90%, such as at least 95% sequence identity to SEQ ID NO: 2
   d. a polypeptide having at least 90%, such as at least 95% sequence identity to the hybrid polypeptide of SEQ ID NO: 3
   e. a polypeptide having at least 90%, such as at least 95% sequence identity to the hybrid polypeptide of SEQ ID NO: 3 wherein the hybrid polypeptide comprises a substitution, a deletion or an insertion in one of more of positions: 48, 49, 107, 156, 181, 190, 197, 201, 209 and/or 264 when using SEQ ID NO: 3 for numbering;
   f. a polypeptide having at least 90%, such as at least 95% sequence identity to SEQ ID NO: 4;
   g. a polypeptide having at least 90%, such as at least 95% sequence identity to SEQ ID NO: 4 or a variant thereof wherein the polypeptide comprises a substitution, a deletion or an insertion in one of more of positions: 181, 182, 183, 184, 195, 206, 212, 216 and/or 269 when using SEQ ID NO: 4 for numbering;
   h. a polypeptide having at least 90%, such as at least 95% sequence identity to SEQ ID NO: 5, SEQ ID NO: 6 or SEQ ID NO: 7;
   i. a polypeptide having at least 90%, such as at least 95% sequence identity to SEQ ID NO: 5, SEQ ID NO: 6 or SEQ ID NO: 7 or a variant thereof wherein the polypeptide comprises a substitution, a deletion or an insertion in one of more of positions: 140, 183, 184 195, 206, 243, 260, 304 and/or 476 when using SEQ ID NO: 5, SEQ ID NO: 6 or SEQ ID NO: 7 for numbering;
   j. a polypeptide having at least 90%, such as at least 95% sequence identity to SEQ ID NO: 8;
   k. a polypeptide having at least 90%, such as at least 95% sequence identity to SEQ ID NO: 9;
   l. a polypeptide having at least 90%, such as at least 95% sequence identity to SEQ ID NO: 9 or a variant thereof wherein the polypeptide comprises a substitution, a deletion or an insertion in one of more of positions: 176, 177, 178, 179, 190, 201, 207, 211 and/or 264 when using SEQ ID NO: 9 for numbering;
   m. a polypeptide having at least 90%, such as at least 95% sequence identity to SEQ ID NO: 10;
   n. a polypeptide having at least 90%, such as at least 95% sequence identity to SEQ ID NO: 10 or a variant thereof wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475 when using SEQ ID NO: 10 for numbering;
   o. a polypeptide having at least 90%, such as at least 95% sequence identity to SEQ ID NO: 11;
   p. a polypeptide having at least 90%, such as at least 95% sequence identity to SEQ ID NO: 11 or a variant thereof wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484 when using SEQ ID NO: 11 for numbering; and
   q. a polypeptide having at least 80%, at least 85%, at least 90%, such as at least 95% sequence identity to SEQ ID NO: 18
23. Use according to any of the preceding use paragraphs, wherein the protease is a serine protease or a metalloprotease, preferably an alkaline microbial protease or a trypsin-like protease.
24. Use according to any of the preceding use paragraphs, wherein the protease is selected from the group consisting of Bacillus, e.g., subtilisin Novo, subtilisin Carlsberg, subtilisin 309, subtilisin 147, subtilisin 168, trypsin of bovine origin, trypsin of porcine origin and Fusarium protease.
25. Use according to any of the preceding use paragraphs, wherein the protease is selected from the group consisting of:
   a. a polypeptide having at least 90%, such as at least 95% sequence identity to SEQ ID NO: 12;
   b. a polypeptide having at least 90%, such as at least 95% sequence identity to SEQ ID NO: 12 or a variant thereof wherein the polypeptide comprises a substitution in one or more of positions: 9, 15, 27, 36, 68, 76, 87, 95, 96, 97, 98, 99, 100, 101, 102, 103, 104, 106, 118, 120, 123, 128, 129, 130, 160, 167, 170, 194, 195, 218, 222, 232, 235, 236, 245, 248, 252 and/or 274 using BPN′ numbering;
   c. a polypeptide having at least 90%, such as at least 95% sequence identity to SEQ ID NO: 13; and
   d. a polypeptide having at least 90%, such as at least 95% sequence identity SEQ ID NO: 13 or a variant thereof wherein the polypeptide comprises a substitution in one or more of positions: 3, 4, 99, 101, 103, 104, 159, 194, 199, 205 and/or 217 when using SEQ ID NO: 13 for numbering.
26. Use according to any of the preceding use paragraphs, wherein the polypeptide having at least 90%, such as at least 95% sequence identity to SEQ ID NO: 12 comprises the following modifications: Y167A+R170S+A194P or Y161A+R164S+A188P when using SEQ ID NO: 12 for numbering.
27. Use according to any of the preceding use paragraphs, wherein the mannanase is selected from the group consisting of:
   a. a polypeptide having at least 80%, such as at least 85%, at least 90% or at least 95% sequence identity to amino acids 31 to 330 of SEQ ID NO: 14;
   b. a polypeptide having at least 80%, such as at least 85%, at least 90% or at least 95% sequence identity to amino acids 32 to 334 of SEQ ID NO: 15;
   c. a polypeptide having at least 80%, such as at least 85%, at least 90% or at least 95% sequence identity to amino acids 33 to 331 of SEQ ID NO: 16; and
   d. a polypeptide having at least 80%, such as at least 85%, at least 90% or at least 95% sequence identity to amino acids 68 to 369 of SEQ ID NO: 17.
28. Use according to any of the preceding use paragraphs comprising at least the following enzymes:
   a. An alpha-amylase having at least 90%, such as at least 95% sequence identity to SEQ ID NO: 9 or SEQ ID NO: 11 and a protease having at least 90%, such as at least 95% sequence identity to SEQ ID NO: 12;
   b. An alpha-amylase having at least 90%, such as at least 95% sequence identity to SEQ ID NO: 9 or SEQ ID NO: 11; a protease having at least 90%, such as at least 95% sequence identity to SEQ ID NO: 12 and a mannanase having at least 80%, such as at least 85%, at least 90%, or at least 95% sequence identity to amino acids 31 to 330 of SEQ ID NO: 14;
   c. An alpha-amylase having at least 90%, such as at least 95% sequence identity to SEQ ID NO: 9 or SEQ ID NO: 11 and a mannanase having at least 80%, such as at least 85%, at least 90%, or at least 95% sequence identity to amino acids 31 to 330 of SEQ ID NO: 14;
   d. An alpha-amylase having at least 90%, such as at least 95% sequence identity to SEQ ID NO: 21 or SEQ ID NO: 22 and a protease having at least 90%, such as at least 95% sequence identity to SEQ ID NO: 12;
   e. An alpha-amylase having at least 90%, such as at least 95% sequence identity to SEQ ID NO: 21 or SEQ ID NO: 22; a protease having at least 90%, such as at least 95% sequence identity to SEQ ID NO: 12 and a mannanase having at least 80%, such as at least 85%, at least 90%, or at least 95% sequence identity to amino acids 31 to 330 of SEQ ID NO: 14;
   f. An alpha-amylase having at least 90%, such as at least 95% sequence identity to SEQ ID NO: 21 or SEQ ID NO: 22 and a mannanase having at least 80%, such as at least 85%, at least 90%, or at least 95% sequence identity to amino acids 31 to 330 of SEQ ID NO: 14;
   g. a protease having at least 90%, such as at least 95% sequence identity to SEQ ID NO: 12 and a mannanase having at least 80%, such as at least 85%, at least 90%, or at least 95% sequence identity to amino acids 31 to 330 of SEQ ID NO: 14;
   h. a polypeptide having at least 80%, at least 85%, at least 90%, such as at least 95% sequence identity to SEQ ID NO: 18 and a protease having at least 90%, such as at least 95% sequence identity to SEQ ID NO: 12;
   i. a polypeptide having at least 80%, at least 85%, at least 90%, such as at least 95% sequence identity to SEQ ID NO: 18, a protease having at least 90%, such as at least 95% sequence identity to SEQ ID NO: 12; a mannanase having at least 80%, such as at least 85%, at least 90% or at least 95% sequence identity to amino acids 31 to 330 of SEQ ID NO: 14;
   j. a polypeptide having at least 80%, at least 85%, at least 90%, such as at least 95% sequence identity to SEQ ID NO: 18 and a mannanase having at least 80%, such as at least 85%, at least 90%, or at least 95% sequence identity to amino acids 31 to 330 of SEQ ID NO: 14;
   k. a polypeptide having at least 80%, at least 85%, at least 90%, such as at least 95% sequence identity to SEQ ID NO: 18 and a protease having at least 90%, such as at least 95% sequence identity to SEQ ID NO: 12 comprising the following modifications: Y167A+R170S+A194P or Y161A+R164S+A188P when using SEQ ID NO: 12 for numbering.
   l. a polypeptide having at least 80%, at least 85%, at least 90%, such as at least 95% sequence identity to SEQ ID NO: 18, a protease having at least 90%, such as at least 95% sequence identity to SEQ ID NO: 12 comprising the following modifications: Y167A+R170S+A194P or Y161A+R164S+A188P when using SEQ ID NO: 12 for numbering, and a mannanase having at least 80%, such as at least 85%, at least 90% or at least 95% sequence identity to amino acids 31 to 330 of SEQ ID NO: 14;
   m. a protease having at least 90%, such as at least 95% sequence identity to SEQ ID NO: 12 comprising the following modifications: Y167A+R170S+A194P or Y161A+R164S+A188P when using SEQ ID NO: 12 for numbering and a mannanase having at least 80%, such as at least 85%, at least 90%, or at least 95% sequence identity to amino acids 31 to 330 of SEQ ID NO: 14.
29. Use according to any of the preceding use paragraphs, wherein the said enzymes are present in a concentration of 0.001-200 mg of protein, such as 0.005-100 mg of protein, preferably 0.01-50 mg of protein, more preferably 0.05-20 mg of protein, even more preferably 0.1-10 mg of protein per liter of wash liquor.
30. Use according to any of the preceding use paragraphs, wherein one or more further enzymes are used, wherein the further enzymes are selected from the group consisting of proteases, lipases, cutinases, alpha-amylases, carbohydrases, cellulases, pectinases, mannanases, arabinases, galactanases, xylanases and oxidases.
31. Use according to any of the preceding use paragraphs, wherein the item is a textile, a fabric, a hard surface or a dishware.
32. A cleaning or laundering method for cleaning or laundering an item comprising the steps of:
   a. Exposing the item to a wash liquor comprising a detergent composition according to any of paragraphs 1-16 or exposing the item to two or more enzymes selected from the group consisting of
      i. protease and alpha-amylase,
      ii. protease and mannanase,
      iii. mannanase and alpha-amylase and
      iv. protease, mannanase and alpha-amylase,
   b. Completing at least one wash cycle; and
   c. optionally rinsing the item
wherein the item is a textile, a fabric, a dishware or a hard surface.
33. Method according to paragraph 32, wherein the pH of the wash liquor is in the range of 7 to 10.5.
34. Method according to any of the preceding method paragraphs, wherein the pH of the wash liquor is in the range of 7 to 9, in the range of 7 to 8 or in the range of 7 to 7.5.
35. Method according to any of the preceding method paragraphs, wherein the temperature of the wash liquor is in the range of 5° C. to 95° C., or in the range of 10° C. to 80° C., in the range of 10° C. to 70° C., in the range of 10° C. to 60° C., in the range of 10° C. to 50° C., in the range of 15° C. to 40° C., in the range of 20° C. to 30° C. or in the range of 10° C. to 15° C.
36. Method according to any of the preceding method paragraphs, wherein the temperature of the wash liquor is 15° C.
37. Method according to any of the preceding paragraphs, wherein the said enzymes are present in a concentration of 0.001-200 mg of protein, such as 0.005-100 mg of protein, preferably 0.01-50 mg of protein, more preferably 0.05-20 mg of protein, even more preferably 0.1-10 mg of protein per liter of wash liquor.
38. Method according to any of the preceding method paragraphs, wherein the method further comprises draining of the wash liquor or part of the wash liquor after completion of a wash cycle.
39. Method according to any of the preceding method paragraphs, wherein the item is exposed to the wash liquor during a first and optionally a second or a third wash cycle.
40. Method according to any of the preceding method paragraphs, wherein the item is rinsed after being exposed to the wash liquor.
41. Method according to any of the preceding method paragraphs, wherein the item is rinsed with water or with water comprising a conditioner.
42. Method according to any of the preceding method paragraphs, wherein the item is textile and/or fabric.
43. Method according to any of paragraphs 34-44, wherein the item is a hard surface.

### EXAMPLES

Laundry detergent composition

| Ingredient | Amount (in wt %) |
|---|---|
| Anionic detersive surfactant (such as alkyl benzene sulphonate, alkyl | from 8 wt % to |
| ethoxylated sulphate and mixtures | 15 wt % thereof) |
| Non-ionic detersive surfactant (such as alkyl ethoxylated alcohol) | from 0.5 wt % |
| | to 4 wt % |
| Cationic detersive surfactant (such as quaternary ammonium compounds) | from 0 to 4 wt % |
| Other detersive surfactant (such as zwiterionic detersive surfactants, | from 0 wt % to |
| amphoteric surfactants and mixtures thereof) | 4 wt % |
| Carboxylate polymer (such as co-polymers of maleic acid and acrylic acid) | from 1 wt % to |
| | 4 wt % |
| Polyethylene glycol polymer (such as a polyethylene glycol polymer comprising | from 0.5 wt % to |
| poly vinyl acetate side chains) | 4 wt % |
| Polyester soil release polymer (such as Repel-o-tex from and/or Texcare | 0.1 to 2 wt % |
| polymers) | |
| Cellulosic polymer (such as carboxymethyl cellulose, methyl cellulose and | from 0.5 wt % to |
| combinations thereof) | 2 wt % |
| Other polymer (such as amine polymers, dye transfer inhibitor polymers, | from 0 wt % to |
| hexamethylenediamine derivative polymers, and mixtures thereof) | 4 wt % |
| Zeolite builder and phosphate builder (such as zeolite 4A and/or sodium | from 0 wt % to |
| tripolyphosphate) | 4 wt % |
| Other builder (such as sodium citrate and/or citric acid) | from 0 wt % to |
| | 3 wt % |
| Carbonate salt (such as sodium carbonate and/or sodium bicarbonate) | from 15 wt % to |
| | 30 wt % |
| Silicate salt (such as sodium silicate) | from 0 wt % to |
| | 10 wt % |
| Filler (such as sodium sulphate and/or bio-fillers) | from 10 wt % to |
| | 40 wt % |
| Source of available oxygen (such as sodium percarbonate) | from 10 wt % to |
| | 20 wt % |
| Bleach activator (such as tetraacetylethylene diamine (TAED) and/or | from 2 wt % to |
| nonanoyloxybenzenesulphonate (NOBS) | 8 wt % |
| Bleach catalyst (such as oxaziridinium-based bleach catalyst and/or transition | from 0 wt % to |
| metal bleach catalyst) | 0.1 wt % |
| Other bleach (such as reducing bleach and/or pre- formed peracid) | from 0 wt % to |
| | 10 wt % |
| Chelant (such as ethylenediamine-N′N′-disuccinic acid (EDDS) and/or | from 0.2 wt % to |
| hydroxyethane diphosphonic acid(HEDP) | 1 wt % |
| Photobleach (such as zinc and/or aluminium sulphonated phthalocyanine) | from 0 wt % to |
| | 0.1 wt % |
| Hueing agent (such as direct violet 99, acid red 52, acid blue 80, direct violet 9, | from 0 wt % to |
| solvent violet 13 and any combination thereof) | 1 wt % |
| Brightener (such as brightener 15 and/or brightener 49) | from 0.1 wt % to |
| | 0.4 wt % |
| Protease (such as Savinase, Savinase Ultra, Purafect, FN3, FN4 and any | from 0.1 wt % to |
| combination thereof) | 0.4 wt % |
| Amylase (such as Termamyl, Termamyl ultra Natalase, Optisize, Stainzyme, | from 0.05 wt % to |
| Stainzyme Plus, and any combination thereof) | 0.2 wt % |
| Cellulase (such as Carezyme and/or Celluclean) | from 0.05 wt % to |
| | 0.2 wt % |
| Lipase (such as Lipex, Lipolex, Lipoclean and any combination thereof) | from 0.2 to 1 wt % |
| Other enzyme (such as xyloglucanase, cutinase, pectate lyase, mannanase, | from 0 wt % to |
| bleaching enzyme) | 2 wt % |
| Fabric softener (such as montmorillonite clay and/or polydimethylsiloxane | from 0 wt % to |
| (PDMS) | 4 wt % |
| Flocculant (such as polyethylene oxide) | from 0 wt % to |
| | 1 wt % |
| Suds suppressor (such as silicone and/or fatty acid) | from 0 wt % to |
| | 0.1 wt % |
| Perfume (such as perfume microcapsule, spray-on perfume, starch | from 0.1 wt % to |
| encapsulated perfume accords, perfume loaded zeolite, and any combination | 1 wt % |
| thereof) | |
| Aesthetics (such as coloured soap rings and/or coloured speckles/noodles) | from 0 wt % to |
| | 1 wt % |
| Miscellaneous | balance |
| Ingredient | Amount |
| Carboxyl group-containing polymer (comprising from about 60% to about 70% | from about 0.5 wt % |
| by mass of an acrylic acid-based monomer (A); and from about 30% to about | to about 1.5 wt % |
| 40%) by mass of a sulfonic acid group-containing monomer (B); and wherein | |
| the average molecular weight is from about 23,000 to about 50,000 preferably | |
| in the range of from about 25,000 to about 38,000 as described in | |
| WO2014032269. | |
| Amylase (Stainzyme Plus(R), having an enzyme activity of 14 mg active | from about 0.1 wt % |
| enzyme/g) | to about 0.5 wt % |
| Anionic detersive surfactant (such as alkyl benzene sulphonate, alkyl | from about 8 wt % |
| ethoxylated sulphate and mixtures thereof) | to about 15 wt % |
| Non-ionic detersive surfactant (such as alkyl ethoxylated alcohol) | from about 0.5 wt % |
| | to 4 wt % |
| Cationic detersive surfactant (such as quaternary ammonium compounds) | from about 0 wt % |
| | to about 4 wt % |
| Other detersive surfactant (such as zwiterionic detersive surfactants, | from about 0 wt % |
| amphoteric surfactants and mixtures thereof) | to 4 wt % |
| Carboxylate polymer (such as co-polymers of maleic acid and acrylic acid) | from about 1 wt % |
| | to about 4 wt % |
| Polyethylene glycol polymer (such as a polyethylene glycol polymer comprising | from about 0 wt % |
| poly vinyl acetate side chains) | to about 4 wt % |
| Polyester soil release polymer (such as Repel-O- Tex(R) and/or Texcare(R) | from about 0.1 wt % |
| polymers) | to about 2 wt % |
| Cellulosic polymer (such as carboxymethyl cellulose, methyl cellulose and | from about 0.5 wt % |
| combinations thereof) | to about 2 wt % |
| Other polymer (such as amine polymers, dye transfer inhibitor polymers, | from about 0 wt % |
| hexamethylenediamine derivative polymers, and mixtures thereof) | to about 4 wt % |
| Zeolite builder and phosphate builder (such as zeolite 4A and/or sodium | from about 0 wt % |
| tripolyphosphate) | to about 4 wt % |
| Other builder (such as sodium citrate and/or citric acid) | from about 0 wt % |
| | to about 3 wt % |
| Carbonate salt (such as sodium carbonate and/or sodium bicarbonate) | from about 15 t % |
| | to about 30 wt % |
| Silicate salt (such as sodium silicate) | from about 0 wt % |
| | to about 10 wt % |
| Filler (such as sodium sulphate and/or bio-fillers) | from about 10 wt % |
| | to about 40 wt % |
| Source of available oxygen (such as sodium percarbonate) | from about 10 wt % |
| | to about 20 wt % |
| Bleach activator (such as tetraacetylethylene diamine (TAED) and/or | from about 2 wt % |
| nonanoyloxybenzenesulphonate (NOBS) | to about 8 wt % |
| Bleach catalyst (such as oxaziridinium-based bleach catalyst and/or transition | from about 0 wt % |
| metal bleach catalyst) | to about 0.1 wt % |
| Other bleach (such as reducing bleach and/or pre formed peracid) | from about 0 wt % |
| | to about 10 wt % |
| Chelant (such as ethylenediamine-N′N′-disuccinic acid (EDDS) and/or | from about 0.2 wt % |
| hydroxyethane diphosphonic acid (HEDP) | to about 1 wt % |
| Photobleach (such as zinc and/or aluminium sulphonated phthalocyanine) | from about 0 wt % |
| | to about 0.1 wt % |
| Hueing agent (such as direct violet 99, acid red 52, acid blue 80, direct violet 9, | from about 0 wt % |
| solvent violet 13 and any combination thereof) | to about 0.5 wt % |
| Brightener (such as brightener 15 and/or brightener 49) | from about 0.1 wt % |
| | to about 0.4 wt % |
| Protease (such as Savinase, Polarzyme, Purafect, FN3, FN4 and any | from about 0.1 wt % |
| combination thereof, typically having an enzyme activity of from about 20 mg to | to about 1.5 wt % |
| about 100 mg active enzyme/g) | |
| Amylase (such as Termamyl(R), Termamyl Ultra(R), Natalase(R), Optisize HT | from about 0.05 wt % |
| Plus(R), Powerase(R), Stainzyme(R) and any combination thereof, typically | to about 0.2 wt % |
| having an enzyme activity of from about 10 mg to about 50 mg active enzyme/g) | |
| Cellulase (such as Carezyme(R), Celluzyme(R) and/or Celluclean(R), typically | from about 0.05 wt % |
| having an enzyme activity of about from 10 to 50 mg active enzyme/g) | to 0.5 wt % |
| Lipase (such as Lipex(R), Lipolex(R), Lipoclean(R) and any combination | from about 0.2 wt % |
| thereof, typically having an enzyme activity of from about 10 mg to about 50 | to about 1 wt % |
| mg active enzyme/g) | |
| Other enzyme (such as xyloglucanase (e.g., Whitezyme(R)), cutinase, pectate | from 0 wt % to |
| lyase, mannanase, bleaching enzyme, typically having an enzyme activity | 2 wt % |
| of from about 10 mg to about 50 mg active enzyme/g) | |
| Fabric softener (such as montmorillonite clay and/or polydimethylsiloxane | from 0 wt % to |
| (PDMS)) | 15 wt % |
| Flocculant (such as polyethylene oxide) | from 0 wt % to |
| | 1 wt % |
| Suds suppressor (such as silicone and/or fatty acid) | from 0 wt % to |
| | 0.1 wt % |
| Perfume (such as perfume microcapsule, spray-on perfume, starch | from 0.1 wt % to |
| encapsulated perfume accords, perfume loaded zeolite, and any combination thereof) | 1 wt % |
| Aesthetics (such as colored soap rings and/or colored speckles/noodles) | from 0 wt % to |
| | 1 wt % |
| Miscellaneous | Balance |

All enzyme levels expressed as rug active enzyme protein per 100 g detergent composition.

Surfactant ingredients can be obtained from BASF, Ludwigshafen, Germany (Lutensol®); Shell Chemicals, London, UK; Stepan, Northfield, Ill., USA; Huntsman, Huntsman, Salt Lake City, Utah, USA; Clariant, Sulzbach, Germany (Praepagen®).
Sodium tripolyphosphate can be obtained from Rhodia, Paris, France.
Zeolite can be obtained from Industrial Zeolite (UK) Ltd, Grays, Essex, UK.
Citric acid and sodium citrate can be obtained from Jungbunzlauer, Basel, Switzerland.
NOBS is sodium nonanoyloxybenzenesulfonate, supplied by Eastman, Batesville, Ark., USA.
TAED is tetraacetylethylenediamine, supplied under the Peractive® brand name by Clariant GmbH, Sulzbach, Germany.
Sodium carbonate and sodium bicarbonate can be obtained from Solvay, Brussels, Belgium.
Polyacrylate, polyacrylate/maleate copolymers can be obtained from BASF, Ludwigshafen, Germany.
Repel-O-Tex® can be obtained from Rhodia, Paris, France.
Texcare® can be obtained from Clariant, Sulzbach, Germany. Sodium percarbonate and sodium carbonate can be obtained from Solvay, Houston, Tex., USA.
Na salt of Ethylenediamine-N,N′-disuccinic acid, (S,S) isomer (EDDS) was supplied by Octel, Ellesmere Port, UK.
Hydroxy ethane di phosphonate (HEDP) was supplied by Dow Chemical, Midland, Mich., USA.

### Enzymes Savinase®, Savinase® Ultra, Stainzyme® Plus, Lipex®, Lipolex®, Lipoclean®, Celluclean®, Carezyme®, Natalase®, Stainzyme®, Stainzyme® Plus, Termamyl®,

Termamyl® ultra, and Mannaway® can be obtained from Novozymes, Bagsvaerd, Denmark.

Enzymes Purafect®, FN3, FN4 and Optisize can be obtained from Genencor International Inc., Palo Alto, Calif., US.
Direct violet 9 and 99 can be obtained from BASF DE, Ludwigshafen, Germany.
Solvent violet 13 can be obtained from Ningbo Lixing Chemical Co., Ltd. Ningbo, Zhejiang, China.

Brighteners can be obtained from Ciba Specialty Chemicals, Basel, Switzerland. All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Wash Assays

### Terg-O-Tometer (TOM) Wash Assay

The Tergo-To-Meter (TOM) is a medium scale model wash system that can be applied to test 12 different wash conditions simultaneously. A TOM is basically a large temperature controlled water bath with up to 16 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

The TOM model wash system is mainly used in medium scale testing of detergents and enzymes at US or LA/AP wash conditions. In a TOM experiment, factors such as the ballast to soil ratio and the fabric to wash liquor ratio can be varied. Therefore, the TOM provides the link between small scale experiments, such as AMSA and mini-wash, and the more time consuming full scale experiments in top loader washing machines.

Equipment: The water bath with 16 steel beakers and 1 rotating arm per beaker with capacity of 500 or 1200 mL of detergent solution. Temperature ranges from 5 to 80° C.?. The water bath has to be filled up with deionised water. Rotational speed can be set up to 40 to 200 rpm/min?.

Set temperature in the Terg-O-Tometer and start the rotation in the water bath. Wait for the temperature to adjust (tolerance is +/−0.1° C.)

All beakers shall be clean and without traces of prior test material.

Prepare wash solution with desired amount of detergent, temperature and water hardness in a bucket. Let detergent dissolve during magnet stirring for 10 min. Wash solution shall be used within 30 to 60 min after preparation.

Add 1000 ml wash solution into a TOM beaker

Start agitation at 120 rpm and optionally add enzymes to the beaker.

Sprinkle the swatches into the beaker and then the ballast load.

Time measurement start when the swatches and ballast are added to the beaker.

Wash for 20 minutes

Stop agitation

Transfer the wash load from TOM beaker to a sieve and rinse with cold tap water

Separate the soil swatches from the ballast load. The soil swatches are transferred to a 5 L beaker with cold tap water under running water. Keep the ballast load separately for the coming inactivation.

Set the timer to 5 minutes.

Press gently the water out by hand and place the test swatches on a tray covered with a paper. Add another paper on top of the swatches.

Let the swatches dry overnight and then measure at the Color Eye as described below.

### Evaluation of Stains

Wash performance is expressed as a delta remission value (ΔRem). After washing and rinsing the swatches were spread out flat and allowed to air dry at room temperature overnight. All washes are evaluated the day after the wash. Light reflectance evaluations of the swatches were done using a Macbeth Color Eye 7000 reflectance spectrophotometer with very small aperture. The measurements were made without UV in the incident light and remission at 460 nm was extracted. Measurements were made on unwashed and washed swatches. The test swatch to be measured was placed on top of another swatch of same type and colour (twin swatch). With only one swatch of each kind per beaker, a swatch from a replicate wash was used in this way. Remission values for individual swatches were calculated by subtracting the remission value of the unwashed swatch from the remission value of the washed swatch. The total wash performance for each stained swatch set was calculated as the sum of individual ΔRem.

Calculating the enzyme effect is done by taking the measurements from washed swatches with enzymes and subtract with the measurements from washed without enzyme for each stain. The total enzyme performance is calculated as the sum of individual ΔRem_{enzyme}.

### Example 1

### Construction of the Alpha Amylase of SEQ ID NO: 18

Construction of a hybrid between the A and B domain from an alpha amylase having the amino acid sequence of SEQ ID NO: 7 (the first amylase) and the C domain from an amylase having the amino acid sequence of SEQ ID NO: 9 (the second amylase).

Based on 3D structural alignment of the two amylases, amino acid no 1 to amino acid no 399 of the first amylase are defined as domain A and B, and amino acid 398 to amino acid no. 483 of the second amylase are defined as the C domain. A synthetic DNA fragment (SEQ ID NO: 19) coding for part of the A domain from the first amylase and the C domain from the second amylase was designed and purchased from an external vendor. In addition to combining fragments of the two different amylases, the following stabilizing substitutions were designed into the synthetic amylase gene: H183*, G184*, I405L, A428T, A421H, A422P.

The new amylase gene consisting of a gene fragment encoding the A and B domain of the first amylase and the C-domain of the second amylase coded by the synthetic amylase gene fragment was constructed by triple SOE (splicing by overlap extension) PCR method. A DNA fragment was amplified from an expression clone of the first amylase integrated into the Pel logi of B. subtilis by the primer set CA375 (SEQ ID NO: 23)+CA378 (SEQ ID NO: 26). The synthetic gene fragment was amplified and extended by PCR using the primer set CA376 (SEQ ID NO: 24)+LBei1302 (SEQ ID NO: 27). A third fragment including the terminator and a downstream Pel logi was amplified by the primer set CA377 (SEQ ID NO: 25)+LBei1303 (SEQ ID NO: 28). The three fragments were finally assembled by triple SOE and the derived PCR fragment was transformed into a suitable B. subtilis host and the gene integrated into the Bacillus subtilis chromosome by homologous recombination into the pectate lyase (pel) locus. The gene coding for chloramphenicol acetyltransferase was used as maker (as described in (Diderichsen et al., 1993, Plasmid 30: 312-315). Chloramphenicol resistant clones were analyzed by DNA sequencing to verify the correct DNA sequence of the construct. The resulting amylase consisting of the A and B domain from the first alpha amylase and the C domain from the second amylase, and having the deletions of H183* and G184* and the substitutions of I405L, A428T, A421H, A422P is the amylase of SEQ ID NO: 18.

### Example 2

The experimental conditions for the experiments are specified in Table 1, Table 2, Table 3 and Table 4 below. The synergy effect is shown in Table 5.

TABLE 1
Experimental conditions for TOM wash

| | | |
|---|---|---|
| Washing time | 20 | min |
| Agitation speed | 120 | rpm |
| Temperature | 20° | C. |
| Water hardness | 15° | dH |

| | |
|---|---|
| Ca²⁺:Mg²⁺:CO₃²⁻ | 4:1:7.5 |
| ratio | |
| Ballast | No ballast |

TABLE 2
Formulations for detergent

| POWDER DETERGENT | Origin | Dosage (g/L) |
|---|---|---|
| Model A, liquid detergent | EU | 3.3 |

Liquid Model A detergent is composed of: AEO (11%), soap (5.5%), AEOS (7%), LAS (12%), polycarboxylates (0.2%), DTMPA (0.2%), Sodium citrate (2%), TEA (3%), Glycerol (2%), Ethanol (3%). MPG (6%), water (40.6%), NaOH (2%), Formate (1%).

TABLE 3
enzymes in detergent

| | | Dosage |
|---|---|---|
| | | mg enzyme |
| Composition | | protein/L |
| Number | enzyme | (mgEP/L) |
| 1 | Protease: SEQ ID NO: 12 with | 0.6 mgEP/L |
| | modifications: Y167A + R170S + A194P | |
| 2 | Amylase: SEQ ID NO: 18 | 0.1 mgEP/L |
| 3 | Mannanase: SEQ ID NO: 14, | 0.01 mgEP/L |

| | |
|---|---|
| 4 | Protease 0.6 mgEP/L + Amylase 0.1 mgEP/L |
| 5 | Protease 0.6 mgEP/L + Mannanase 0.01 mgEP/L |
| 6 | Amylase 0.1 mgEP/L + Mannanase 0.01 mgEP/L |
| 8 | Protease 0.6 mgEP/L + Amylase 0.1 mgEP/L + Mannanase |
| | 0.01 mgEP/L |

TABLE 4
Swatches with stains used

| | | | | Number |
|---|---|---|---|---|
| Producer | Code | Name | size | of swatch in wash |
| CFT | CS-06 | salad dressing | 5 × 5 cm | 2 |
| EMPA | EMPA165 | chocolate pudding | 5 × 5 cm | 2 |

Water hardness was adjusted by addition of CaCl2, MgCl2 and NaHCO3 to the test system. After washing, the swatches were gently rinsed under running tap water for 10 min.

After the rinse, the swatches were dried lying horizontally in a dark room over night at room temperature.

The performance of the enzyme is evaluated by measuring the remission of the textile swatches using the ColorEye at 460 nm (Apparatus model: Largo, CE7000). The synergy effect is shown in Table 5 and 6 below.

TABLE 5
synergy effect on salad dressing.

| | delta Rem enzyme remission | | | | | | | |
|---|---|---|---|---|---|---|---|---|
| | value Calculated and measured | Compo- | Compo- | Compo- | Compo- | Compo- | Compo- | Compo- |
| Row | C-S-06 salad dressing | sition 1 | sition 2 | sition 3 | sition 4 | sition 5 | sition 6 | sition 7 |
| 1 | Individual enzyme effects - | 1.65 | 0.85 | 2.07 | x | x | x | x |
| | measured as described | | | | | | | |
| 2 | Sum of individual enzyme effects - | x | x | x | 2.50 | 3.72 | 2.92 | 4.57 |
| | calculated from the data row 1 | | | | | | | |
| 3 | Effects of enzymes in combination - | x | x | x | 3.49 | 4.40 | 6.63 | 9.61 |
| | measured as described | | | | | | | |
| 4 | Synergy effects - calculated as | x | x | x | 0.99 | 0.68 | 3.72 | 5.04 |
| | difference between row 2 and row 3. | | | | | | | |

TABLE 6
synergy effect on chocolate pudding

| | delta Rem enzyme remission | | | | | | | |
|---|---|---|---|---|---|---|---|---|
| | value Calculated and measured | Compo- | Compo- | Compo- | Compo- | Compo- | Compo- | Compo- |
| Row | EMPA 165 chocolate pudding | sition 1 | sition 2 | sition 3 | sition 4 | sition 5 | sition 6 | sition 7 |
| 1 | Individual enzyme effects - | 1.02 | 0.87 | 4.97 | x | x | x | x |
| | measured as described | | | | | | | |
| 2 | Sum of individual enzyme effects - | x | x | x | 1.89 | 5.99 | 5.83 | 6.86 |
| | calculated from the data row 1 | | | | | | | |
| 3 | Effects of enzymes in combination - | x | x | x | 5.10 | 6.05 | 11.21 | 14.19 |
| | measured as described | | | | | | | |
| 4 | Synergy effects - calculated as | x | x | x | 3.21 | 0.06 | 5.38 | 7.33 |
| | difference between row 2 and row 3. | | | | | | | |

### Example 3

The experimental conditions for the experiments are specified in Table 1, Table 2, Table 3 and Table 4 below. The synergy effect is shown in Table 5.

TABLE 1
Experimental conditions for TOM wash

| | |
|---|---|
| Washing time | 20 min |
| Agitation speed | 120 rpm |
| Temperature | 20° C. |
| Water hardness | 15°dH |
| Ca²⁺:Mg²⁺:CO₃²⁻ ratio | 4:1:7.5 |
| Ballast | No ballast |

TABLE 2
Formulations for detergent

| POWDER DETERGENT | Origin | Dosage (g/L) |
|---|---|---|
| Model A, liquid detergent | EU | 3.3 |

Liquid Model A detergent is composed of: AEO (11%), soap (5.5%), AEOS (7%), LAS (12%), polycarboxylates (0.2%), DTMPA (0.2%), Sodium citrate (2%), TEA (3%), Glycerol (2%), Ethanol (3%). MPG (6%), water (40.6%), NaOH (2%), Formate (1%).

TABLE 3
enzymes in detergent

| | | Dosage |
|---|---|---|
| | | mg enzyme |
| Composition | | protein/L |
| Number | enzyme | (mgEP/L) |
| 1 | Protease: SEQ ID NO: 12, including | 0.6 mgEP/L |
| | protease inhibitor | |
| 2 | Amylase: SEQ ID NO: 18 | 0.1 mgEP/L |
| 3 | Mannanase: SEQ ID NO: 14, | 0.01 mgEP/L |

| | |
|---|---|
| 4 | Protease 0.6 mgEP/L + Amylase 0.1 mgEP/L |
| 5 | Protease 0.6 mgEP/L + Mannanase 0.01 mgEP/L |
| 6 | Amylase 0.1 mgEP/L + Mannanase 0.01 mgEP/L |
| 7 | Protease 0.6 mgEP/L + Amylase 0.1 mgEP/L + |
| | Mannanase 0.01 mgEP/L |

Protease inhibitor is: L-Alaninamide, N-[(phenylmethoxy)carbonyl]glycyl-N-[2-hydroxy-1-[(4-hydroxyphenyl)methyl]-2-sulfoethyl]-, sodium salt (1:1).

TABLE 4
Swatches with stains used

| | | | | Number |
|---|---|---|---|---|
| Producer | Code | Name | size | of swatch in wash |
| CFT | CS-06 | salad dressing | 5 × 5 cm | 2 |
| EMPA | EMPA165 | chocolate pudding | 5 × 5 cm | 2 |

Water hardness was adjusted by addition of CaCl2, MgCl2 and NaHCO3 to the test system. After washing, the swatches were gently rinsed under running tap water for 10 min.

After the rinse, the swatches were dried lying horizontally in a dark room over night at room temperature.

The performance of the enzyme is evaluated by measuring the remission of the textile swatches using the ColorEye at 460 nm (Apparatus model: Largo, CE7000). The synergy effect is shown in Table 5 and 6 below.

TABLE 5
synergy effect on salad dressing.

| | delta Rem enzyme remission | | | | | | | |
|---|---|---|---|---|---|---|---|---|
| | value Calculated and measured | Compo- | Compo- | Compo- | Compo- | Compo- | Compo- | Compo- |
| ROw | C-S-06 salad dressing | sition 1 | sition 2 | sition 3 | sition 4 | sition 5 | sition 6 | sition 7 |
| 1 | Individual enzyme effects - | 0.25 | 0 | 1.85 | x | x | x | x |
| | measured as described | | | | | | | |
| 2 | Sum of individual enzyme effects - | x | x | x | 0.25 | 2.09 | 1.85 | 2.09 |
| | calculated from the data row 1 | | | | | | | |
| 3 | Effects of enzymes in combination - | x | x | x | 2.28 | 3.24 | 4.81 | 8.37 |
| | measured as described | | | | | | | |
| 4 | Synergy effects - calculated as | x | x | x | 2.03 | 1.15 | 2.96 | 6.28 |
| | difference between row 2 and row 3. | | | | | | | |

TABLE 6
synergy effect on chocolate pudding

| | delta Rem enzyme remission | | | | | | | |
|---|---|---|---|---|---|---|---|---|
| | value Calculated and measured | Compo- | Compo- | Compo- | Compo- | Compo- | Compo- | Compo- |
| Row | EMPA 165 chocolate pudding | sition 1 | sition 2 | sition 3 | sition 4 | sition 5 | sition 6 | sition 7 |
| 1 | Individual enzyme effects - | 1.16 | 5.81 | 4.65 | x | x | x | x |
| | measured as described | | | | | | | |
| 2 | Sum of individual enzyme effects - | x | x | x | 6.97 | 5.82 | 10.46 | 11.63 |
| | calculated from the data row 1 | | | | | | | |
| 3 | Effects of enzymes in combination - | x | x | x | 6.09 | 5.18 | 14.73 | 13.67 |
| | measured as described | | | | | | | |
| 4 | Synergy effects - calculated as | x | x | x | −0.88 | −0.64 | 4.27 | 2.04 |
| | difference between row 2 and row 3. | | | | | | | |

### Example 4

The experimental conditions for the experiments are specified in Table 1, Table 2, Table 3 and Table 4 below. The synergy effect is shown in Table 5.

TABLE 1
Experimental conditions for TOM wash

| | |
|---|---|
| Washing time | 20 min |
| Agitation speed | 120 rpm |
| Temperature | 20° C. |
| Water hardness | 15°dH |
| Ca²⁺:Mg²⁺:CO₃²⁻ ratio | 4:1:7.5 |
| Ballast | No ballast |

TABLE 2
Formulations for detergent

| POWDER DETERGENT | Origin | Dosage (g/L) |
|---|---|---|
| Model A, liquid detergent | EU | 3.3 |

Liquid Model A detergent is composed of: AEO (11%), soap (5.5%), AEOS (7%), LAS (12%), polycarboxylates (0.2%), DTMPA (0.2%), Sodium citrate (2%), TEA (3%), Glycerol (2%), Ethanol (3%). MPG (6%), water (40.6%), NaOH (2%), Formate (1%).

TABLE 3
enzymes in detergent

| | | Dosage |
|---|---|---|
| Composition | | mg enzyme protein/ |
| Number | enzyme | L (mgEP/L) |
| 1 | Protease: SEQ ID NO: 12 with | 0.6 mgEP/L |
| | modifications: Y161A + R164S + | |
| | A188P, including protease inhibitor | |
| 2 | Amylase: SEQ ID NO: 21 | 0.1 mgEP/L |
| 3 | Mannanase: SEQ ID NO: 14, | 0.01 mgEP/L |

| | |
|---|---|
| 4 | Protease 0.6 mgEP/L + Amylase 0.1 mgEP/L |
| 5 | Amylase 0.1 mgEP/L + Mannanase 0.01 mgEP/L |
| 6 | Protease 0.6 mgEP/L + Amylase 0.1 mgEP/L + Mannanase |
| | 0.01 mgEP/L |

Protease inhibitor is L-Alaninamide, N-[(phenylmethoxy)carbonyl]glycyl-N-[2-hydroxy-1-[(4-hydroxyphenyl)methyl]-2-sulfoethyl]-, sodium salt (1:1).

TABLE 4
Swatches with stains used

| | | | | Number |
|---|---|---|---|---|
| Producer | Code | Name | size | of swatch in wash |
| CFT | CS-06 | salad dressing | 5 × 5 cm | 2 |
| EMPA | EMPA165 | chocolate pudding | 5 × 5 cm | 2 |

Water hardness was adjusted by addition of CaCl2, MgCl2 and NaHCO3 to the test system. After washing, the swatches were gently rinsed under running tap water for 10 min.

After the rinse, the swatches were dried lying horizontally in a dark room over night at room temperature.

The performance of the enzyme is evaluated by measuring the remission of the textile swatches using the ColorEye at 460 nm (Apparatus model: Largo, CE7000). The synergy effect is shown in Table 5 and 6 below.

TABLE 5
synergy effect on salad dressing.

| | delta Rem enzyme remission | | | | | | |
|---|---|---|---|---|---|---|---|
| | value Calculated and measured | Compo- | Compo- | Compo- | Compo- | Compo- | Compo- |
| Row | C-S-06 salad dressing | sition 1 | sition 2 | sition 3 | sition 4 | sition 5 | sition 6 |
| 1 | Individual enzyme effects - | 0.96 | 0 | 1.85 | x | x | x |
| | measured as described | | | | | | |
| 2 | Sum of individual enzyme effects - | x | x | x | 0.96 | 1.85 | 2.81 |
| | calculated from the data row 1 | | | | | | |
| 3 | Effects of enzymes in combination - | x | x | x | 2.72 | 3.69 | 8.37 |
| | measured as described | | | | | | |
| 4 | Synergy effects - calculated as | x | x | x | 1.76 | 1.84 | 5.56 |
| | difference between row 2 and row 3. | | | | | | |

TABLE 6
synergy effect on chocolate pudding

| | delta Rem enzyme remission | | | | | | |
|---|---|---|---|---|---|---|---|
| | value Calculated and measured | Compo- | Compo- | Compo- | Compo- | Compo- | Compo- |
| Row | EMPA 165 chocolate pudding | sition 1 | sition 2 | sition 3 | sition 4 | sition 5 | sition 6 |
| 1 | Individual enzyme effects - | 2.38 | 5.48 | 4.65 | x | x | x |
| | measured as described | | | | | | |
| 2 | Sum of individual enzyme effects - | x | x | x | 7.86 | 10.14 | 12.52 |
| | calculated from the data row 1 | | | | | | |
| 3 | Effects of enzymes in combination - | x | x | x | 5.04 | 12.69 | 14.34 |
| | measured as described | | | | | | |
| 4 | Synergy effects - calculated as | x | x | x | −2.82 | 2.55 | 1.82 |
| | difference between row 2 and row 3. | | | | | | |

### Example 5

The experimental conditions for the experiments are specified in Table 1, Table 2, Table 3 and Table 4 below. The synergy effect is shown in Table 5.

TABLE 1
Experimental conditions for TOM wash

| | |
|---|---|
| Washing time | 20 min |
| Agitation speed | 120 rpm |
| Temperature | 20° C. |
| Water hardness | 15° dH |
| Ca²⁺:Mg²⁺:CO₃²⁻ ratio | 4:1:7.5 |
| Ballast | No ballast |

TABLE 2
Formulations for detergent

| POWDER DETERGENT | Origin | Dosage (g/L) |
|---|---|---|
| Model A, liquid detergent | EU | 3.3 |

Liquid Model A detergent is composed of: AEO (11%), soap (5.5%), AEOS (7%), LAS (12%), polycarboxylates (0.2%), DTMPA (0.2%), Sodium citrate (2%), TEA (3%), Glycerol (2%), Ethanol (3%). MPG (6%), water (40.6%), NaOH (2%), Formate (1%).

TABLE 3
enzymes in detergent

| | | Dosage |
|---|---|---|
| | | mg enzyme |
| Composition | | protein/ |
| Number | enzyme | L (mgEP/L) |
| 1 | Protease: SEQ ID NO: 12 with | 0.6 mgEP/L |
| | modifications: Y161A + R164S + | |
| | A188P, including protease inhibitor | |
| 2 | Amylase: SEQ ID NO: 18 | 0.1 mgEP/L |
| 3 | Mannanase: SEQ ID NO: 14, | 0.01 mgEP/L |

| | |
|---|---|
| 4 | Protease 0.6 mgEP/L + Amylase 0.1 mgEP/L |
| 5 | Protease 0.6 mgEP/L + Mannanase 0.01 mgEP/L |
| 6 | Amylase 0.1 mgEP/L + Mannanase 0.01 mgEP/L |
| 8 | Protease 0.6 mgEP/L + Amylase 0.1 mgEP/L + |
| | Mannanase 0.01 mgEP/L |

Protease inhibitor is L-Alaninamide, N-[(phenylmethoxy)carbonyl]glycyl-N-[2-hydroxy-1-[(4-hydroxyphenyl)methyl]-2-sulfoethyl]-, sodium salt (1:1).

TABLE 4
Swatches with stains used

| | | | | Number |
|---|---|---|---|---|
| Producer | Code | Name | size | of swatch in wash |
| CFT | CS-06 | salad dressing | 5 × 5 cm | 2 |
| EMPA | EMPA165 | chocolate pudding | 5 × 5 cm | 2 |

Water hardness was adjusted by addition of CaCl2, MgCl2 and NaHCO3 to the test system. After washing, the swatches were gently rinsed under running tap water for 10 min.

After the rinse, the swatches were dried lying horizontally in a dark room over night at room temperature.

The performance of the enzyme is evaluated by measuring the remission of the textile swatches using the ColorEye at 460 nm (Apparatus model: Largo, CE7000). The synergy effect is shown in Table 5 and 6 below.

TABLE 5
synergy effect on salad dressing.

| | delta Rem enzyme remission | | | | | | | |
|---|---|---|---|---|---|---|---|---|
| | value Calculated and measured | Compo- | Compo- | Compo- | Compo- | Compo- | Compo- | Compo- |
| Row | C-S-06 salad dressing | sition 1 | sition 2 | sition 3 | sition 4 | sition 5 | sition 6 | sition 7 |
| 1 | Individual enzyme effects - | 1.65 | 0.85 | 2.07 | x | x | x | x |
| | measured as described | | | | | | | |
| 2 | Sum of individual enzyme effects - | x | x | x | 2.50 | 3.72 | 2.92 | 4.57 |
| | calculated from the data row 1 | | | | | | | |
| 3 | Effects of enzymes in combination - | x | x | x | 3.49 | 4.40 | 6.63 | 9.61 |
| | measured as described | | | | | | | |
| 4 | Synergy effects - calculated as | x | x | x | 0.99 | 0.68 | 3.72 | 5.04 |
| | difference between row 2 and row 3. | | | | | | | |

TABLE 6
synergy effect on chocolate pudding

| | delta Rem enzyme remission | | | | | | | |
|---|---|---|---|---|---|---|---|---|
| | value Calculated and measured | Compo- | Compo- | Compo- | Compo- | Compo- | Compo- | Compo- |
| Row | EMPA 165 chocolate pudding | sition 1 | sition 2 | sition 3 | sition 4 | sition 5 | sition 6 | sition 7 |
| 1 | Individual enzyme effects - | 1.02 | 0.87 | 4.97 | x | x | x | x |
| | measured as described | | | | | | | |
| 2 | Sum of individual enzyme effects - | x | x | x | 1.89 | 5.99 | 5.83 | 6.86 |
| | calculated from the data row 1 | | | | | | | |
| 3 | Effects of enzymes in combination - | x | x | x | 5.10 | 6.05 | 11.21 | 14.19 |
| | measured as described | | | | | | | |
| 4 | Synergy effects - calculated as | x | x | x | 3.21 | 0.06 | 5.38 | 7.33 |
| | difference between row 2 and row 3. | | | | | | | |

1-15. (canceled)

16. A detergent composition comprising a detergent adjunct ingredient and two or more enzymes selected from the group consisting of:
a. protease and alpha-amylase,
b. protease and mannanase,
c. mannanase and alpha-amylase, and
d. protease, mannanase and alpha-amylase.

17. The detergent composition of claim 16, wherein the polypeptide having alpha-amylase activity is selected from the group consisting of:
a. a polypeptide having at least 90% sequence identity to SEQ ID NO: 1;
b. a polypeptide having at least 90% sequence identity to SEQ ID NO: 1 or a variant thereof wherein the polypeptide comprises a substitution in one or more of positions: 15, 23, 105, 106, 124, 128, 133, 154, 156, 178, 179, 181, 188, 190, 197, 201, 202, 207, 208, 209, 211, 243, 264, 304, 305, 391, 408, and/or 444 when using SEQ ID NO: 1 for numbering;
c. a polypeptide having at least 90% sequence identity to SEQ ID NO: 2;
d. a polypeptide having at least 90% sequence identity to the hybrid polypeptide of SEQ ID NO: 3;
e. a polypeptide having at least 90% sequence identity to the hybrid polypeptide of SEQ ID NO: 3 wherein the hybrid polypeptide comprises a substitution, a deletion or an insertion in one of more of positions: 48, 49, 107, 156, 181, 190, 197, 201, 209 and/or 264, when using SEQ ID NO: 3 for numbering;
f. a polypeptide having at least 90% sequence identity to SEQ ID NO: 4;
g. a polypeptide having at least 90% sequence identity to SEQ ID NO: 4 or a variant thereof wherein the polypeptide comprises a substitution, a deletion or an insertion in one of more of positions: 181, 182, 183, 184, 195, 206, 212, 216 and/or 269 when using SEQ ID NO: 4 for numbering;
h. a polypeptide having at least 90% sequence identity to SEQ ID NO: 5, SEQ ID NO: 6 or SEQ ID NO: 7;
i. a polypeptide having at least 90% sequence identity to SEQ ID NO: 5, SEQ ID NO: 6 or SEQ ID NO: 7 or a variant thereof wherein the polypeptide comprises a substitution, a deletion or an insertion in one of more of positions: 140, 183, 184 195, 206, 243, 260, 304 and/or 476 when using SEQ ID NO: 5 for numbering;
j. a polypeptide having at least 90% sequence identity to SEQ ID NO: 8;
k. a polypeptide having at least 90% sequence identity to SEQ ID NO: 9;
l. a polypeptide having at least 90% sequence identity to SEQ ID NO: 9 or a variant thereof wherein the polypeptide comprises a substitution, a deletion or an insertion in one of more of positions: 176, 177, 178, 179, 190, 201, 207, 211 and/or 264 when using SEQ ID NO: 9 for numbering;
m. a polypeptide having at least 90% sequence identity to SEQ ID NO: 10;
n. a polypeptide having at least 90% sequence identity to SEQ ID NO: 10 or a variant thereof wherein the polypeptide comprises a substitution, a deletion or an insertion in one of more of positions: 87, 98, 125, 128, 131, 165, 178, 180, 181, 182, 183, 201, 202, 225, 243, 272, 282, 305, 309, 319, 320, 359, 444 and/or 475 when using SEQ ID NO: 10 for numbering;
o. a polypeptide having at least 90% sequence identity to SEQ ID NO: 11;
p. a polypeptide having at least 90% sequence identity to SEQ ID NO: 11 or a variant thereof wherein the polypeptide comprises a substitution, a deletion or an insertion in one of more of positions: 28, 118, 174; 181, 182, 183, 184, 186, 189, 195, 202, 298, 299, 302, 303, 306, 310, 314; 320, 324, 345, 396, 400, 439, 444, 445, 446, 449, 458, 471 and/or 484;
q. a polypeptide having at least 80% sequence identity to SEQ ID NO: 18;
r. a polypeptide having at least 80% sequence identity to SEQ ID NO: 20;
s. a polypeptide having at least 80% sequence identity to SEQ ID NO: 21; and
t. a polypeptide having at least 80% sequence identity to SEQ ID NO: 22;
the protease is selected from the group consisting of:
a. a polypeptide having at least 90% sequence identity to SEQ ID NO: 12;
b. a polypeptide having at least 90% sequence identity to SEQ ID NO: 12 or a variant thereof wherein the polypeptide comprises a substitution in one or more of positions: 9, 15, 27, 36, 68, 76, 87, 95, 96, 97, 98, 99, 100, 101, 102, 103, 104, 106, 118, 120, 123, 128, 129, 130, 160, 167, 170, 194, 195, 218, 222, 232, 235, 236, 245, 248, 252 and/or 274 using BPN′ numbering;
c. a polypeptide having at least 90% sequence identity to SEQ ID NO: 13; and
d. a polypeptide having at least 90% sequence identity SEQ ID NO: 13 or a variant thereof wherein the polypeptide comprises a substitution in one or more of positions: 3, 4, 99, 101, 103, 104, 159, 194, 199, 205 and/or 217;
e. a polypeptide having at least 90% sequence identity to SEQ ID NO: 12 comprises the following modifications: Y167A+R170S+A194P Y161A+R164S+A188P when using SEQ ID NO: 12 for numbering; and
the mannanase is selected from the group consisting of:
a. a polypeptide having at least 80% sequence identity to amino acids 31 to 330 of SEQ ID NO: 14;
b. a polypeptide having at least 80% sequence identity to amino acids 32 to 334 of SEQ ID NO: 15;
c. a polypeptide having at least 80% sequence identity to amino acids 33 to 331 of SEQ ID NO: 16; and
d. a polypeptide having at least 80% sequence identity to amino acids 68 to 369 of SEQ ID NO: 17.

18. The detergent composition of claim 16, comprising at least the following enzymes:
a. an alpha-amylase having at least 90% sequence identity to SEQ ID NO: 9 or SEQ ID NO: 11 and a protease having at least 90% sequence identity to SEQ ID NO: 12;
b. an alpha-amylase having at least 90% sequence identity to SEQ ID NO: 9 or SEQ ID NO: 11; a protease having at least 90% sequence identity to SEQ ID NO: 12 and a mannanase having at least 80% sequence identity to amino acids 31 to 330 of SEQ ID NO: 14;
c. an alpha-amylase having at least 90% sequence identity to SEQ ID NO: 9 or SEQ ID NO: 11 and a mannanase having at least 80% sequence identity to amino acids 31 to 330 of SEQ ID NO: 14;
d. an alpha-amylase having at least 90% sequence identity to SEQ ID NO: 21 or SEQ ID NO: 22 and a protease having at least 90% sequence identity to SEQ ID NO: 12;
e. an alpha-amylase having at least 90% sequence identity to SEQ ID NO: 21 or SEQ ID NO: 22; a protease having at least 90% sequence identity to SEQ ID NO: 12 and a mannanase having at least 80% sequence identity to amino acids 31 to 330 of SEQ ID NO: 14;
f. an alpha-amylase having at least 90% sequence identity to SEQ ID NO: 21 or SEQ ID NO: 22 and a mannanase having at least 80% sequence identity to amino acids 31 to 330 of SEQ ID NO: 14;
g. a protease having at least 90% sequence identity to SEQ ID NO: 12 and a mannanase having at least 80% sequence identity to amino acids 31 to 330 of SEQ ID NO: 14;
h. a polypeptide having at least 80% sequence identity to SEQ ID NO: 18 and a protease having at least 90% sequence identity to SEQ ID NO: 12;
i. a polypeptide having at least 80% sequence identity to SEQ ID NO: 18, a protease having at least 90% sequence identity to SEQ ID NO: 12; a mannanase having at least 80% sequence identity to amino acids 31 to 330 of SEQ ID NO: 14;
j. a polypeptide having at least 80% sequence identity to SEQ ID NO: 18 and a mannanase having at least 80% sequence identity to amino acids 31 to 330 of SEQ ID NO: 14;
k. a polypeptide having at least 80% sequence identity to SEQ ID NO: 18 and a protease having at least 90% sequence identity to SEQ ID NO: 12 comprising the following modifications: Y167A+R170S+A194P or Y161A+R164S+A188P when using SEQ ID NO: 12 for numbering;
l. a polypeptide having at least 80% sequence identity to SEQ ID NO: 18, a protease having at least 90% sequence identity to SEQ ID NO: 12 comprising the following modifications: Y167A+R170S+A194P or Y161A+R164S+A188P when using SEQ ID NO: 12 for numbering, and a mannanase having at least 80% sequence identity to amino acids 31 to 330 of SEQ ID NO: 14; or
m. a protease having at least 90% sequence identity to SEQ ID NO: 12 comprising the following modifications: Y167A+R170S+A194P or Y161A+R164S+A188P when using SEQ ID NO: 12 for numbering and a mannanase having at least 80% sequence identity to amino acids 31 to 330 of SEQ ID NO: 14.

19. The detergent composition of claim 16, wherein the detergent adjunct ingredient is selected from the group consisting of surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids and/or pigments.

20. A method for cleaning or laundering an item comprising the steps of:
a. exposing the item to a wash liquor comprising a detergent composition of claim 16 or exposing the item to two or more enzymes selected from the group consisting of
i. protease and alpha-amylase,
ii. protease and mannanase,
iii. mannanase and alpha-amylase and
iv. protease, mannanase and alpha-amylase; and
b. completing at least one wash cycle;
wherein the item is a textile, a fabric, a dishware or a hard surface.

21. The method of claim 20, further comprising rinsing the item.

22. The method of claim 20, wherein the pH of the wash liquor is in the range of 7 to 10.5.

23. The method of claim 20, wherein the temperature of the wash liquor is in the range of 5° C. to 95° C.

24. The method of claim 20, wherein the temperature of the wash liquor is in the range of 15° C. to 40° C.

25. The method of claim 20, wherein the enzymes are present in a concentration of 0.001-200 mg of protein per liter of wash liquor.

26. The method of claim 20, wherein the enzymes are present in a concentration of 0.05-20 mg of protein per liter of wash liquor.


SEQUENCE LISTING (19 sequences)

SEQ ID NO: 1 - protein, 483 aa, Bacillus licheniformis [sequence omitted]

SEQ ID NO: 2 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGGTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 3 - protein, 481 aa, Artificial sequence
  Synthetic construct
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 4 - protein, 485 aa, Bacillus species
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 5 - protein, 485 aa, Bacillus species
HHNGTNGTMMQYFEWYLPNDGNHWNRLRDDAANLKSKGITAVWIPPAWKGTSQNDVGYGAYDLYDLGEFNQKGTVRTKYGTRNQLQAAVTSLKNNGIQVYGDVVMNHKGGADGTEIVNAVEVNRSNRNQETSGEYAIEAWTKFDFPGRGNNHSSFKWRWYHFDGTDWDQSRQLQNKIYKFRGTGKAWDWEVDTENGNYDYLMYADVDMDHPEVIHELRNWGVWYTNTLNLDGFRIDAVKHIKYSFTRDWLTHVRNTTGKPMFAVAEFWKNDLGAIENYLNKTSWNHSVFDVPLHYNLYNASNSGGYYDMRNILNGSVVQKHPTHAVTFVDNHDSQPGEALESFVQQWFKPLAYALVLTREQGYPSVFYGDYYGIPTHGVPAMKSKIDPLLQARQTFAYGTQHDYFDHHDIIGWTREGNSSHPNSGLATIMSDGPGGNKWMYVGKNKAGQVWRDITGNRTGTVTINADGWGNFSVNGGSVSVWVKQ

SEQ ID NO: 6 - protein, 485 aa, Bacillus species
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 7 - protein, 485 aa, Bacillus species; identical to SEQ ID NO: 4

SEQ ID NO: 8 - protein, 485 aa, Bacillus species
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLAYALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 9 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 10 - protein, 484 aa, Bacillus species
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 11 - protein, 485 aa, Bacillus species
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 12 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 13 - protein, 269 aa, Bacillus Lentus
AQTVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 14 - protein, 299 aa, Bacillus species
ANSGFYVSGTTLYDANGNPFVMRGINHGHAWYKDQATTAIEGIANTGANTVRIVLSDGGQWTKDDIHTVRNLISLAEDNHLVAVLEVHDATGYDSIASLNRAVDYWIEMRSALIGKEDTVIINIANEWFGSWEGDAWADGYKQAIPRLRNAGLNHTLMVDAAGWGQFPQSIHDYGREVFNADPQRNTMFSIHMYEYAGGNASQVRTNIDRVLNQDLALVIGEFGHRHTNGDVDEATIMSYSEQRGVGWLAWSWKGNGPEWEYLDLSNDWAGNNLTAWGNTIVNGPYGLRETSRLSTVFQ

SEQ ID NO: 15 - protein, 493 aa, Bacillus agaradhaerens
MKKKLSQIYHLIICTLIISVGIMGITTSPSAASTGFYVDGNTLYDANGQPFVMRGINHGHAWYKDTASTAIPAIAEQGANTIRIVLSDGGQWEKDDIDTIREVIELAEQNKMVAVVEVHDATGRDSRSDLNRAVDYWIEMKDALIGKEDTVIINIANEWYGSWDGSAWADGYIDVIPKLRDAGLTHTLMVDAAGWGQYPQSIHDYGQDVFNADPLKNTMFSIHMYEYAGGDANTVRSNIDRVIDQDLALVIGEFGHRHTDGDVDEDTILSYSEETGTGWLAWSWKGNSTEWDYLDLSEDWAGQHLTDWGNRIVHGADGLQETSKPSTVFTDDNGGHPEPPTATTLYDFEGSTQGWHGSNVTGGPWSVTEWGASGNYSLKADVNLTSNSSHELYSEQSRNLHGYSQLNATVRHANWGNPGNGMNARLYVKTGSDYTWHSGPFTRINSSNSGTTLSFDLNNIENSHHVREIGVQFSAADNSSGQTALYVDNVTLR

SEQ ID NO: 16 - protein, 331 aa, Bacillus species
VYKLTHTYFVALICSILIFAGVLNTSSSQAEAHHSGFHVNGTTLYDANGNPFVMRGINHGHAWFKQELETSMRGISQTGANTIRVVLSNGQRWQKDDRNMVASVISLAEQHQMIAVLEVHDATGSNNFSDLQAAVDYWIEMKDVLQGKEDIVIINIANEWYGAWDGGAWARGYQNAIRQLRNAGLSHTFMVDAAGYGQYPQSVVDYGQEVLNADPQRNTMFSVHMYEYAGGDANTVRRNIDSILSQNLALVIGEFGHWHYDGDVDEDTILSYSQQRNVGWLAWSWHGNSEGVEYLDLSNDFAGNRLTWWGDRIVNGPNGIRQTSKRSSVFQ

SEQ ID NO: 17 - protein, 369 aa, Bacillus species
MRSMKLLFAMFILVFSSFTFNLVVAQASGHGQMHKVPWAPQAEAPGKTAENGVWDKVRNNPGKANPPAGKVNGFYIDGTTLYDANGKPFVMRGINHGHSWYKPHIETAMEAIADTGANSIRVVLSDGQQWTKDDVDEVAKIISLAEKHSLVAALEVHDALGTDDIEPLLKTVDYWIEIKDALIGKEDKVIINISNEWFGSWSSEGWADGYKKAIPLLREAGLKHTLMVDAAGWGQFPRSIHEKGLEVFNSDPLKNTMFSIHMYEWAAGNPQQVKDNIDGVLEKNLAVVIGEFGHHHYGRDVAVDTILSHSEKYDVGWLAWSWHGNSGGVEYLDLATDFSGTQLTEWGERIVHGPNGLKETSEIVSVYKK

SEQ ID NO: 18 - protein, 483 aa, Artificial sequence
  synthetic construct
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYLDHPDVIGWTREGDSSHPKSGLATLITDGPGGSKRMYAGLKNAGETWYDITGNRSDTVKIGSDGWGEFHVNDGSVSIYVQK

SEQ ID NO: 19 - DNA, 588 bp, Artificial sequence
  Synthetic construct
gatgtgccgttacattataatctttataatgcatcaaaaagcggagggaactatgatatgcgaaacatatttaatggaacggttgttcaacgacatccaagtcatgctgtaacatttgttgataatcatgattcgcagcctgaagaagcattagaatcttttgttgaagaatggtttaaaccattagcgtatgcgcttacattaacgcgtgaacaaggatacccttctgtattttacggagattattatgggattccaacacatggagtgccagcaatgagatcaaaaatcgatccgattttagaagcacgtcaaaagtatgcatacggaccccagcacgattatcttgaccacccggatgtgatcggatggacgagggaaggtgacagctcccatcctaaatcaggtttggccactttaatcacggacggacccggcggatcaaagcggatgtatgccggcctgaaaaatgccggcgagacatggtatgacataacgggcaaccgttcagatactgtaaaaatcggatctgacggctggggagagtttcatgtaaacgatgggtccgtctccatttatgttcagaaa